SEQUENCE LISTING (34 sequences)

SEQ ID NO: 1 - DNA, 10 bp, synthetic construct
  source 1..10: mol_type = other DNA
cctatccctg

SEQ ID NO: 2 - DNA, 64 bp, synthetic construct
  source 1..64: mol_type = other DNA
  misc_feature 39: note = Unique Hand mix ratio 25% per nucleotide
aaaaatcgtcggcagcgtcagatgtgtataagagacagnnnnnnnnnntgaggccctatccctg

SEQ ID NO: 3 - DNA, 10 bp, synthetic construct
  source 1..10: mol_type = other DNA
agtctatatg

SEQ ID NO: 4 - DNA, 65 bp, synthetic construct
  source 1..65: mol_type = other DNA
  misc_feature 17: note = Unique Hand mix ratio 25% per nucleotide
agtctatatgcccgatnnnnnnnnnnctgtctcttatacacatctccgagcccacgagacaaaaa

SEQ ID NO: 5 - DNA, 10 bp, synthetic construct
  source 1..10: mol_type = other DNA
tatccctgag

SEQ ID NO: 6 - DNA, 64 bp, synthetic construct
  source 1..64: mol_type = other DNA
  misc_feature 39: note = Unique Hand mix ratio 25% per nucleotide
aaaaatcgtcggcagcgtcagatgtgtataagagacagnnnnnnnnnncgtgggtatccctgag

SEQ ID NO: 7 - DNA, 10 bp, synthetic construct
  source 1..10: mol_type = other DNA
tctatatgat

SEQ ID NO: 8 - DNA, 65 bp, synthetic construct
  source 1..65: mol_type = other DNA
  misc_feature 17: note = Unique Hand mix ratio 25% per nucleotide
tctatatgatgacctcnnnnnnnnnnctgtctcttatacacatctccgagcccacgagacaaaaa

SEQ ID NO: 9 - DNA, 10 bp, synthetic construct
  source 1..10: mol_type = other DNA
tatgatgact

SEQ ID NO: 10 - DNA, 64 bp, synthetic construct
  source 1..64: mol_type = other DNA
  misc_feature 39: note = Unique Hand mix ratio 25% per nucleotide
aaaaatcgtcggcagcgtcagatgtgtataagagacagnnnnnnnnnnctgccgtatgatgact

SEQ ID NO: 11 - DNA, 10 bp, synthetic construct
  source 1..10: mol_type = other DNA
atcgtaccct

SEQ ID NO: 12 - DNA, 65 bp, synthetic construct
  source 1..65: mol_type = other DNA
  misc_feature 17: note = Unique Hand mix ratio 25% per nucleotide
atcgtaccctgcgacgnnnnnnnnnnctgtctcttatacacatctccgagcccacgagacaaaaa

SEQ ID NO: 13 - DNA, 10 bp, synthetic construct
  source 1..10: mol_type = other DNA
atgatgacta

SEQ ID NO: 14 - DNA, 64 bp, synthetic construct
  source 1..64: mol_type = other DNA
  misc_feature 39: note = Unique Hand mix ratio 25% per nucleotide
aaaaatcgtcggcagcgtcagatgtgtataagagacagnnnnnnnnnntctgggatgatgacta

SEQ ID NO: 15 - DNA, 10 bp, synthetic construct
  source 1..10: mol_type = other DNA
tcgtaccctg

SEQ ID NO: 16 - DNA, 65 bp, synthetic construct
  source 1..65: mol_type = other DNA
  misc_feature 17: note = Unique Hand mix ratio 25% per nucleotide
tcgtaccctgtagcacnnnnnnnnnnctgtctcttatacacatctccgagcccacgagacaaaaa

SEQ ID NO: 17 - DNA, 10 bp, synthetic construct
  source 1..10: mol_type = other DNA
atccctgagt

SEQ ID NO: 18 - DNA, 64 bp, synthetic construct
  source 1..64: mol_type = other DNA
  misc_feature 39: note = Unique Hand mix ratio 25% per nucleotide
aaaaatcgtcggcagcgtcagatgtgtataagagacagnnnnnnnnnngtctacatccctgagt

SEQ ID NO: 19 - DNA, 10 bp, synthetic construct
  source 1..10: mol_type = other DNA
ctatatgatg

SEQ ID NO: 20 - DNA, 65 bp, synthetic construct
  source 1..65: mol_type = other DNA
  misc_feature 17: note = Unique Hand mix ratio 25% per nucleotide
ctatatgatgtagatannnnnnnnnnctgtctcttatacacatctccgagcccacgagacaaaaa

SEQ ID NO: 21 - DNA, 37 bp, synthetic construct
  source 1..37: mol_type = other DNA
  modified_base order(6,11,14,17,20,22,27,34): mod_base = OTHER; note = Int deoxyUridine
cagggtacgatagtcatcatatagactcagggatagg

SEQ ID NO: 22 - DNA, 19 bp, synthetic construct
  source 1..19: mol_type = other DNA
gtctcgtgggctcggagat

SEQ ID NO: 23 - DNA, 18 bp, synthetic construct
  source 1..18: mol_type = other DNA
tcgtcggcagcgtcagat

SEQ ID NO: 24 - DNA, 129 bp, synthetic construct
  source 1..129: mol_type = other DNA
aaaaatcgtcggcagcgtcagatgtgtataagagacagnnnnnnnnnntgtagagatgactatcgtaccctgcacggtacnnnnnnnnnnctgtctcttatacacatctccgagcccacgagacaaaaa

SEQ ID NO: 25 - protein, 6 aa, synthetic construct
TGAGGC

SEQ ID NO: 26 - protein, 6 aa, synthetic construct
CCCGAT

SEQ ID NO: 27 - protein, 6 aa, synthetic construct
CGTGGG

SEQ ID NO: 28 - protein, 6 aa, synthetic construct
GACCTC

SEQ ID NO: 29 - protein, 6 aa, synthetic construct
CTGCCG

SEQ ID NO: 30 - protein, 6 aa, synthetic construct
GCGACG

SEQ ID NO: 31 - protein, 6 aa, synthetic construct
TCTGGG

SEQ ID NO: 32 - protein, 6 aa, synthetic construct
TAGCAC

SEQ ID NO: 33 - protein, 6 aa, synthetic construct
GTCTAC

SEQ ID NO: 34 - protein, 6 aa, synthetic construct
TAGATA

(19) United States
(12) Patent Application Publication
(10) Pub. No.: US 2025/0180545 A1
(43) Pub. Date: Jun. 5, 2025

(54) A TUNABLE PROXIMITY ASSAY THAT CAN OVERCOME DILUTIONAL NON-LINEARITY

(71) Applicants: CZ Biohub SF, LLC, San Francisco, CA (US); The Board of Trustees of the Leland Stanford Junior University, Stanford, CA (US)

(72) Inventors: Hyongsok Soh, Palo Alto, CA (US); Brandon D. Wilson, Redwood City, CA (US); Sharon Shin Newman, Palo Alto, CA (US); Daniel Mamerow, Saint Louis Park, MN (US)

(21) Appl. No.: 18/729,030

(22) PCT Filed: Feb. 13, 2023
(86) PCT No.: PCT/US2023/062463

§ 371 Date: Jul. 15, 2024

Related U.S. Application Data
(60) Provisional application No. 63/309,981, filed on Feb. 14, 2022.

(51) Int. Cl. G01N 33/53; G01N 33/543

(52) U.S. Cl. CPC G01N 33/5306; G01N 33/54313

(57) ABSTRACT

Provided herein are methods of tuning a proximity reporter assay. The methods include providing multiple series of compositions with first binding agents labeled with nucleic acid molecules, wherein compositions in the series comprise the first and second binding agents at different concentrations from the other compositions in the series, forming a plurality of mixtures by contacting each composition from the series of compositions with the target analytes; performing, for each mixture in the plurality of mixtures, a proximity assay, wherein the assay produces, for each mixture, a signal comprising a nucleic acid product and selecting from each of the series of compositions a single composition comprising binding agents at a concentration that results in signals within four orders of magnitude for the target analytes. The method can be performed for any number of analytes.

### CROSS REFERENCE TO RELATED APPLICATION

This application is a national phase application of PCT Application No. PCT/US2023/062463, filed Feb. 13, 2023, which claims priority to U.S. Provisional Application No. 63/309,981, filed Feb. 14, 2022, the entire contents of which are incorporated herein by reference for all purposes.

### REFERENCE TO AN ELECTRONIC SEQUENCE LISTING

The contents of the electronic sequence listing (110221-1449374-007310US SL.xml; Size: 36,000 bytes; and Date of Creation: Mar. 7, 2023) is herein incorporated by reference in its entirety.

### BACKGROUND

Blood-based quantification of protein biomarkers is a standard tool for the diagnosis, monitoring, and treatment of disease. However, the physiological concentrations of different plasma proteins can vary considerably, spanning over 10 orders of magnitude (Anderson, N. L. & Anderson, N. G. The human plasma proteome: history, character, and diagnostic prospects. Mol. Cell. Proteomics 1, 845-867 (2002))—ranging from low-femtomolar quantity cytokines to plasma proteins at high nanomolar concentrations—and there is no single assay that can quantify both low- and high-abundance proteins simultaneously from a single sample. This is due to an inability to maintain high-resolution signals from high protein concentrations without obscuring the signal from low-abundance targets, and vice versa. For example, physiological concentrations of C-reactive protein (CRP) are approximately a million-fold greater than those of interleukin 6 (IL-6), such that the signal from CRP in a conventional multiplexed assay would overwhelm the signal from IL-6. Current assays work around this problem by preparing samples at different dilutions, such that the analytes of interest reach an appropriate concentration for detection with a given modality. As a consequence, multiplexed detection from a single sample is only feasible for targets that are present at relatively similar concentrations, whereas analytes that differ markedly in their abundance must be characterized separately in assays performed with samples prepared at different dilutions.

Beyond the added complexity of having to split and independently process samples, this approach is also vulnerable to issues arising from non-linear dilution (NLD), which represents one of the most challenging problems in diagnostics. NLD describes the phenomenon wherein measured concentrations of a given analyte deviate greatly from their expected values when measured at different dilutions, directly undermining the ability to meaningfully compare results from multiple assays performed at different dilutions (Bolstad, N., Warren, D. J. & Nustad, K. Heterophilic antibody interference in immunometric assays. Best Pract. Res. Clin. Endocrinol. Metab. 27, 647-661 (2013)). NLD has been attributed to a variety of mechanisms, such as cross-reactivity with endogenous and exogenous antibodies, or matrix effects arising from sample interferents such as lipids, proteins, and salts (Bolstad, N., Warren, D. J. & Nustad, K. Heterophilic antibody interference in immunometric assays. Best Pract. Res. Clin. Endocrinol. Metab. 27, 647-661 (2013); Tate, J. & Ward, G. Interferences in immunoassay. Clin. Biochem. 25, 105-120 (2004); Slavica Dodig. Interferences in quantitative immunochemical methods. Biochem. Medica 19, 50-62 (2009)). The effects of NLD can be dramatic—for example, upon comparing undiluted patient serum samples to those that were diluted 3-fold, Rosenberg-Hasson et al. observed disparate changes in signal ranging widely from 0.61- to 5.45-fold, with the signal from some proteins even increasing upon dilution (Rosenberg-Hasson, Y., Hansmann, L., Liedtke, M., Herschmann, I. & Maecker, H. T. Effects of serum and plasma matrices on multiplex immunoassays. Immunol. Res. 58, 224-233 (2014)). Indeed, the authors observed a proportional change in signal (2.75-3.25-fold) for only 6% of the tested proteins. Perhaps most troublingly, the observed effects of NLD varied not only from analyte to analyte but also from patient sample to patient sample, suggesting that the optimal dilution for each target could vary across samples.

To ensure that NLD is not confounding results, assay panels require spike-and-recovery assessments (Dabah Lugos, M. Assay Linearity and Spike-Recovery Assessment in Optimization protocol for the analysis of Serum Cytokines by Sandwich ELISA Platform. Am. J. Biomed. Sci. Res. 3, 178-183 (2019)) for every new sample processing method and combination of analytes. These assays are designed to confirm that the signal produced by an analyte spiked into both the expected sample matrix and standard diluent are near-identical after dilution. If the magnitude of signal recovery differs, the diluent or dilution factor must be adjusted until the extent of the signal deviation falls within an acceptable range, which is typically +/−20%. But since NLD can arise for a variety of reasons with different combinations of analytes and sample matrices, different adjustment methods must be used to achieve this goal. That makes this approach extremely challenging to scale to larger numbers of analytes and samples, and there is currently no straightforward or universal solution to the NLD problem.

### BRIEF SUMMARY

Provided herein are methods of tuning a proximity reporter assay. The methods include providing a first series of compositions comprising first binding agents comprising a 5′ nucleic acid molecule and second binding agents attached to a 3′ nucleic acid molecule, wherein compositions in the first series comprise the first and second binding agents at different concentrations from the other compositions in the first series, and wherein the first and second binding agents bind a first target analyte; providing a second series of compositions comprising third binding agents comprising a 5′ nucleic acid molecule and fourth binding agents comprising a 3′ nucleic acid molecule, wherein compositions in the second series comprise the third and fourth binding agents at different concentrations from the other compositions in the second series, and wherein the third and fourth binding agents bind a second target analyte; forming a plurality of mixtures by contacting each composition from the first and second series of compositions with the first and second target analytes; performing, for each mixture in the plurality of mixtures, a proximity assay, wherein the assay produces, for each mixture, a signal comprising a nucleic acid product of the 5′ and 3′ nucleic acid molecules, wherein a first signal is produced for the first target and a second signal is produced for the second target; and selecting from each of the first and second series of compositions a single composition comprising binding agents at a concentration that results in first and second signals within four orders of magnitude for the first and second target analytes. The method steps can be repeated one or more times and the method can be performed for any number of analytes. The first series of compositions can include 2, 3, 4, 5, 10, 15, 20, 25, 30, 35, 40, 45, 50 or more compositions. The second series of compositions can include 2, 3, 4, 5, 10, 15, 20, 25, 30, 35, 40, 45, 50 or more compositions. The binding agents can be antibodies, e.g., polyclonal antibodies.

The first and second signals can be within three, two, or one orders of magnitude for the first and second target analytes. The first and second target analytes can be present in the mixtures at a first and second concentration, respectively, and wherein the first and second concentrations vary by at least three orders of magnitude. The first and second concentrations can vary by at least four, five, six, seven, eight, nine, ten, eleven or twelve orders of magnitude.

Optionally, each composition in the second series of compositions further comprises unlabeled antibodies that bind the second target analytes.

The first and/or second target analyte can be a high abundance target analyte or the first and/or second target analyte can be a low or medium abundance target analyte.

The first compositions can further comprise first capture binding agents that bind the first analytes. The first capture binding agents can be attached to a solid surface. The solid surface can be a bead. The second compositions can further comprise second capture binding agents that bind the second analytes. The second capture binding agents can be attached to a solid surface. The solid surface can be a bead.

Optionally, each mixture comprises both of the first and second target analytes.

The proximity assay can be a proximity ligation assay, and wherein the proximity ligation assay comprises ligating the 5′ and 3′ nucleic acids thereby producing the nucleic acid product comprising the 5′ and 3′ nucleic acids. The proximity assay can further comprise amplifying the nucleic acid products. Optionally, the proximity assay further comprises sequencing the amplified nucleic acids products. The amplified nucleic acid products can include a barcode sequence and/or a unique molecular identifier. The barcode can be a target analyte specific barcode.

The method can also include the step of performing a proximity assay using the selected compositions from step (e) by contacting the selected compositions with a biological sample comprising the first and second target analytes and determining the concentrations of the first and second target analytes in the biological sample based on the assay.

Also provided is a method of providing a tuned proximity reporter assay comprising, (a) providing an N series of compositions for N analytes, wherein each series of compositions corresponds to a different analyte, wherein each series comprises compositions comprising first binding agents comprising a 5′ nucleic acid molecule and second binding agents attached to a 3′ nucleic acid molecule for one analyte in the N sets of analytes, wherein the compositions in each series comprise the first and second binding agents at different concentrations from the other compositions in the series; (b) for each N series of compositions, forming a plurality of mixtures by contacting each composition from the series of compositions with the analyte from the N analytes that corresponds to the series of compositions; (c) performing, for each mixture in the plurality of mixtures, a proximity assay, wherein the assay produces, for each mixture, a signal comprising a nucleic acid product of the 5′ and 3′ nucleic acid molecules, wherein each signal corresponds to an analyte; and (d) selecting for each N series of compositions a single composition comprising binding agents at a concentration that results in signals within four orders of magnitude for each analyte in the N analytes, wherein the selected compositions from the N series of compositions provides a multitude of compositions each composition comprising the selected concentration of binding agents for each analyte within the N analytes, thereby providing a tuned proximity reporter assay.

### BRIEF DESCRIPTION OF THE DRAWINGS

FIGS. 1A and 1B are graphs showing the fundamental challenges in simultaneous quantification of high and low concentration analytes. As seen in FIG. 1A, if two targets are present at very different concentrations (e.g., 100-fold) but have approximately the same response curve (top), the strong signal from the high-abundance target (blue) will drown out that of the low-abundance target (yellow). This problem can be addressed by selectively shifting the response curve of the high abundance target to the right (bottom), such that the high-abundance target can be measured in parallel with the low-abundance target without obscuring its signal. As seen in FIG. 1B, high-abundance targets can be challenging to measure without dilution because their concentrations can far exceed the KD of the affinity reagents used to measure them, such that large shifts in concentration will produce a very small signal change (top). However, if the entire response curve is shifted to the right (bottom), then the signal response within the desired concentration range (between the dashed lines) will occur within the steep-sloped portion of the curve rather than the saturated plateau, increasing the quantitative resolution.

FIGS. 2A, 2B and 2C are schematics showing the overview of solid-phase proximity ligation assay (spPLA) with high-throughput sequencing (HTS) readout. In FIG. 2A, target analytes in a sample are bound by pairs of analyte-specific detection antibodies (dAbs). Each pair of dAbs is derived from the same pool of polyclonal antibodies for a given target and is functionalized with a distinct DNA tag. When a target is bound by a pair of dAbs, the DNA tags are ligated to form a ‘reporter sequence’ that can be PCR amplified and analyzed via HTS. The resulting sequence data reveal the relative concentrations of each target in the pool. In FIG. 2B, the two dAb pools are respectively coupled to specially designed DNA tags by their 5′ or 3′ ends. Each DNA tag contains the following elements: a spacer coupled to the antibody, a universal primer adapter for PCR, a unique molecular identifier (UMI) for each antibody molecule, a barcode unique to each antibody pool that encodes the target protein's identity, and a hybridization site that facilitates ligation to its partner dAb in the presence of a complementary ‘hybridization splint’ DNA strand. In FIG. 2C, target molecules are bound by bead-conjugated capture antibodies and subsequently labeled by a pair of dAbs. The dAb-coupled DNA tags are bound by the hybridization splint (purple), which enables the two ends to be ligated. Only correctly paired dAbs enable ligation and production of a DNA reporter (left). If two 3′ probes or two 5′ probes bind a target, no readout is generated (right).

FIGS. 3A, 3B and 3C are graphs showing tuning the detection range of a low abundance target. Two mechanisms are used to precisely tune the output of proximity assays. FIG. 3A shows probe loading entails increasing the dAb concentration in order to shift the equilibrium and increase the fraction of target molecules that are bound to multiple dAbs. This increases the output for all target concentrations (see probe-loaded blue curve versus untuned red curve). The increase is non-uniform and is more pronounced at higher target concentrations, effectively shifting the response curve up and to the right. FIG. 3B shows epitope depletion allows the reporter output to be tuned for a given target. High concentrations of a given analyte can result in saturation of the output from an spPLA reaction. This response curve can be shifted downward by adding a defined concentration of a depletant antibody, which lacks a DNA tag and thus reduces the likelihood of producing a readout for a given dAb binding event (see depleted blue curve versus untuned red curve). Regarding FIG. 3C, since epitope depletion on its own produces a weaker signal, this tuning strategy was combined with probe loading, which has the net effect of shifting the binding curve to the right (see shifted line relative to the untuned curve).

FIGS. 4A-4E are graphs showing tuning the detection range for analytes of varying concentration. FIG. 4A shows tuning spPLA output based on quantitative PCR (qPCR) for a low/medium-abundance protein, GDF-15. Decreasing the dAb concentration from a starting point of 250 pM shifts the response curve downwards. Target output of ˜1 fM reporter DNA at ˜5 pM GDF-15—the log-middle of our target concentration range—with 10 pM dAb was reached. FIG. 4B shows two tuning mechanisms can be utilized in tandem to obtain a sensitive signal response for high-abundance targets like C-reactive protein (CRP). Decreasing probe loading from 250 pM decreases the reporter DNA output at each CRP concentration, but does not improve the assay's quantitative sensitivity over the physiological range. Regarding FIG. 4C, by also applying epitope depletion with unlabeled, competing antibodies, the DNA output was decreased to the desired range and shifted the overall response curve to the right, so that linear detection occurred in the physiologically relevant range. To compensate for the reduced output caused by epitope depletion, 3.93 pM dAb was used, a concentration selected to generate a pre-depletion output of 10 fM reporter. FIGS. 4D and 4E show qPCR readout for four different protein targets before (4D) and after (4E) tuning for each individual analyte. For all panels, error bars represent the standard deviation of three qPCR replicates. For the two lowest concentrations of CRP at 500 nM depletant in panel c, the lower error bar value is negative and therefore cannot be plotted on the log-scale y-axis.

FIGS. 5A and 5B are graphs showing simultaneous quantification of four protein analytes at concentrations spanning seven orders of magnitude. For FIG. 5A, nUMI as a function of analyte concentration from a tuned four-plex assay with six standards (Table 1) containing different concentrations of our four analytes spiked into buffer and chicken serum. The dotted vertical lines represent approximate the log middle of the defined testing range for each target. In FIG. 5B, it was observed close correlation with the measurements in chicken serum versus those measured in buffer (linear fit: m=1.004±0.011, b=0.070±0.011, R\hat{ }2=0.992). For both FIGS. 5A and 5B, all samples were run in triplicate, and the plotted data were derived from a single demultiplexed sequencing run.

FIG. 6 is a graph showing quantification of endogenous concentrations of four biomarkers in human serum samples. Three individual serum samples from healthy male donors were measured for IL-6, IL-1ra, GDF-15, and CRP using our tuned four-plex EVROS assay (left) and three separate Luminex assays (right). The shaded bars represent the approximate physiological ranges expected in healthy donors. The EVROS assay was performed on 5 μL of undiluted serum, whereas the Luminex assays were performed on 25 μL of serum diluted according to the manufacturer's recommendations: 1,000, 100, and 3-fold for CRP, GDF-15, and IL-6/IL-1ra, respectively. Error bars are based on separate measurements of each sample-triplicate for EVROS and duplicate for Luminex.

FIG. 7 is a graph showing effects of non-linear dilution (NLD). In human serum or plasma samples, protein measurements are often effected by ‘dilutional non-linearity’, or the phenomenon that dilution by a certain amount does not result in the same change in signal. Researchers exploring the phenomenon of NLD on the Luminex platform demonstrated that when diluted three-fold, the majority of analytes saw a <3-fold reduction in signal. Only 6% of the tested targets exhibited a proportional reduction in signal, defined as between 2.75-3.25-fold reduction of signal (demarcated by dashed lines). Data obtained and replotted MFI-mean fluorescence intensity.

FIG. 8 is a schematic showing sliding splint design. The hybridization sites on each dAb's DNA tag are designed so that dozens or even hundreds of targets can be detected using a single ligation strand. Tiling the hybridization sites along the ligation strand also ensures specificity; if a cross-reactive binding event occurs, the DNA oligos on the antibodies will not line up and no DNA reporter will be produced.

FIG. 9 is a graph showing UMIs enable high precision at low copy number but offer low precision at high copy number. This trade-off makes the tuning of the DNA output concentration (i.e., the number of ligation events) crucial, so that (1) no single target consumes an overwhelming number of reads, and (2) the concentrations aren't so high that every read has a unique barcode. It was determined the desired output DNA concentration to be ˜1 fM by sequencing different concentrations of control DNA and observing how the resolution of UMIs changes with DNA concentration. The rank-ordered counts of each UMI resulting from different input concentrations of DNA to the PCR reaction can be used to estimate the concentration that confers maximum resolution. As the number of unique molecular bar-codes approaches the read depth of the sequencer, the ability to resolve counts diminishes. The opposite is the case for a small number of bar-codes. It was observed that the ability to resolve 400 pM vs. 8 pM (red/orange) was worse than 1.6 fM vs. 320 aM (blue/purple). An output of 1 fM DNA was identified as optimal, where the DNA output is high enough to minimize Poisson noise but still low enough to be well below the per-target read depth. Triplicates were plotted individually. RPM=reads per million.

FIG. 10 is a graph showing signal drop from epitope depletion. Binding curves from qPCR output for CRP using 0.43 pM probe with 0 and 50 nM of depletant. 50 nM depletant produces a ˜10-15-fold drop in signal. N=3, mean+/−standard deviation.

FIG. 11 is a graph showing EVROS assay specificity. The specificity of our assay was verified by demonstrating that the addition of any target, even very high concentrations of CRP, does not appreciably affect the signals from the other targets due to the requirement of two binding events to generate a signal. Concentrations used were 12,150 pM CRP, 48.6 pM GDF-15, 36.5 pM IL-1ra, and 0.182 pM IL-6. N=3.

FIG. 12 is a graph showing four-parameter logistic curve fit for analyte standards spiked in buffer. The standards in Table 1 were measured with the EVROS assay, and the binding curve was fitted as described in the Methods. The determined parameter fits were used to quantify the remaining samples of unknown concentration.

### DETAILED DESCRIPTION

Provided herein is an assay with a wide dynamic range, which circumvents the problem of NLD by eliminating the need for dilution. The EVROS assay (after the Greek word ε\acute{ν}ρç meaning “range”) entails molecular equalization of the signal output generated by multiple analytes in a solid-phase proximity ligation assay (spPLA), in which the binding of a pair of oligonucleotide-tagged polyclonal detection antibodies (dAbs) to their target produces a barcoded reporter that can be quantified via high-throughput sequencing (HTS). In some embodiments, this dynamic range is achieved by introducing two orthogonal tuning mechanisms that make it possible to independently modulate the reporter output from each analyte to bring these disparate quantitative readouts into a comparable range. As a demonstration, EVROS was used to simultaneously quantify a panel of four proteins whose physiological concentrations range from <20 fM (interleukin-6; IL-6) to >200 nM (C-reactive protein; CRP), achieving an unprecedented dynamic range spanning seven orders of magnitude in a single reaction. Further, EVROS requires a very small sample volume (5 μL of undiluted human serum), making it useful for multiplexed protein quantification across a broad dynamic range in clinical samples.

### Definitions

As used in the disclosure and the appended claims, the singular forms “a,” “an,” and “the” are intended to include the plural forms as well, unless the context clearly indicates otherwise. Also as used herein, “and/or” refers to and encompasses any and all possible combinations of one or more of the associated listed items, as well as the lack of combinations when interpreted in the alternative (“or”).

Unless defined otherwise, all technical and scientific terms used herein have the same meaning as commonly understood by one of ordinary skill in the art to which this invention belongs. All publications mentioned herein are incorporated herein by reference for the purpose of describing and disclosing devices, compositions, formulations, and methodologies which are described in the publication and which might be used in connection with the presently described invention.

As used herein, the term “analyte” or “target analyte” refers to a molecule that can be recognized and bound by a binding agent, e.g., an antibody. A target analyte can be a small molecule (e.g., a small organic molecule), a protein, a peptide, or a nucleic acid (e.g., DNA or RNA).

The terms “nucleic acid,” “nucleic acid sequence,” “nucleic acid molecule” and “polynucleotide” may be used interchangeably herein and refer to a polymeric form of nucleotides of any length, either deoxyribonucleotides or ribonucleotides, or analogs thereof, and may include naturally occurring nucleotides and/or modified nucleotides. Polynucleotides may have any three-dimensional structure, and may perform any function, known or unknown. Non-limiting examples of polynucleotides include a gene, a gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, control regions, isolated RNA of any sequence, nucleic acid probes, and primers. The nucleic acid molecule may be linear or circular. As used herein, the term “oligonucleotide” can refer to a polynucleotide chain, typically less than 200 residues long, most typically between 15 and 100 nucleotides long, but also intended to encompass longer polynucleotide chains. Oligonucleotides can be single- or double-stranded.

As used herein, the terms “5′ nucleic acid” and “3′ nucleic acid” refer to nucleic acids used in a proximity assay as described herein. The reference to 5′ and 3′ is not meant to specify a specific order or end of the nucleic acids. The terms are used to denote the nucleic acids are two separate nucleic acids. As described herein, the 5′ and 3′ nucleic acid molecules are joined to produce a nucleic acid product.

As used herein, the term “binding agent” refers to a molecule capable of binding another molecule. For example, the binding agent can be a polypeptide that binds to another polypeptide or other molecule such as a nucleic acid. By way of another example, the binding agent can be an antibody, an aptamer or a nanobody or combinations thereof.

As used herein, the term “antibody” refers to a protein functionally defined as a binding protein and structurally defined as comprising an amino acid sequence that is recognized by one of skill as being derived from a variable region of an immunoglobulin encoding gene. The term encompasses polyclonal antibodies, monoclonal antibodies, single chain antibodies, multispecific antibodies such as bispecific antibodies, monospecific antibodies, monovalent antibodies, chimeric antibodies, humanized antibodies, and human antibodies. The term “antibody,” as used herein, also includes antibody fragments that retain binding specificity, including but not limited to Fab, F(ab′)2, Fv, scFv, and bivalent scFv. An antibody can consist of one or more polypeptides substantially encoded by immunoglobulin genes or fragments of immunoglobulin genes. The recognized immunoglobulin genes include the kappa, lambda, alpha, gamma, delta, epsilon, and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. The term “antibody,” as used herein, also includes antibody mimetic proteins, for example affibodies, DARPins, and nanobodies. “Antibodies” are a type of “affinity reagent” that also includes aptamers, affimers, knottins and the like.

An exemplary antibody structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms “variable light chain” (VL) and “variable heavy chain” (VH) refer to these light and heavy chains, respectively.

As used herein the term “aptamer” or “aptamer sequence” refers to a nucleic acid having a specific binding affinity for a target, e.g., a target molecule, wherein such target is other than a polynucleotide that binds to the aptamer or aptamer sequence.

As used herein, the term “nanobody” refers to a peptide that binds a target such as an antigen. Typically, nanobodies are small antigen-binding fragments of about 12 to 15 kD in size and a single domain antibody consisting of only one heavy chain variable region. Thus, nanobodies are often derived from the heavy domain of antibodies. Nanobodies can be engineered to bind to a single target or more than one target.

As used herein, the term “barcode” or “BC” refers to a short (typically less than 50 bases, often less than 30 bases) nucleic acid sequence that identifies a property of a polynucleotide. For example, in some cases polynucleotides with the same barcode have a common origin, e.g., are from the same vessel or compartment. In various places in this disclosure there is reference, for clarity, to a barcode sequence and a barcode sequence complement. It will be recognized that in a double-stranded polynucleotide the sequence in both strands is informative and can serve as a barcode. The barcode can be 1-50 nucleotides in length, e.g., 1 to 25, 10 to 25, or 15 to 30 nucleotides in length.

As used herein, the term “unique molecular identifier” (UMI) refers to a nucleic acid sequence that may be used to distinguish individual nucleic acids from one another. Optionally, a UMI sequence contains randomized nucleotides. The UMI can be 1-50 nucleotides in length, e.g., 1 to 25, 10 to 25, or 15 to 30 nucleotides in length.

A “biological sample,” as used herein, generally refers to a bodily tissue or fluid obtained from a human, preferably a mammalian subject. Exemplary subjects include, but are not limited to humans, non-human primates such as monkeys, dogs, cats, mice, rats, cows, horses, camels, goats, and sheep. In some embodiments, the subject is a human. Non-limiting examples of biological samples include blood, blood fractions or blood products (e.g., serum, plasma, platelets, red blood cells, peripheral blood mononuclear cells and the like), sputum or saliva, stool, urine, other biological fluids (e.g., lymph, prostatic fluid, gastric fluid, intestinal fluid, renal fluid, lung fluid, cerebrospinal fluid, and the like). Additionally, solid tissues, for example, tissue biopsies (e.g., subcutaneous fat tissue) may be used. A biological sample may be processed prior to use in a detection assay including dilution, addition of buffer or preservative, concentration, purification, or partial purification.

As used herein, a “proximity assay” (or proximity-based binding assay) refers to an assay that produces a detectable signal when two binding events occur physically close to each other and at the same time. Examples of proximity assays include split reporter-type assays, proximity ligation, and proximity extension assays. See, for example, Darmanis, S. et al. Sensitive plasma protein analysis by microparticle-based proximity ligation assays. Mol. Cell. Proteomics 9, 327-335 (2010) and Ebai, T., Kamali-Moghaddam, M. & Landegren, U. Parallel protein detection by solid-phase Proximity ligation assay with real-time PCR or sequencing. Curr. Protoc. Mol. Biol. 20.10.1-20.10.25 (2015).

As used herein, a “tuned” proximity assay refers to an assay that produces detectable signals that are within four orders of magnitude of each other. For example, in an assay detecting two or more targets, the signals for each target will be within four orders of magnitude of each other. By way of example, if a proximity assay is detecting four target analytes, detection of each target analyte by the proximity assay results in a signal and the signal for each of the four different target analytes are within four orders of magnitude of each of the other signals. Optionally, the signals will be within three, two or one order of magnitude of each other.

As used herein, the term “series” refers to a set, collection or group of things. For example, a “series” of compositions means a set or group of compositions. The series can include one or more compositions. The series, for example, can include 2, 3, 4, 5, 6, 7, 8, 9, 10, 25, 50, 75, 100 or more compositions.

The terms “identical” or percent “identity,” in the context of two or more polypeptide sequences, refer to two or more sequences or subsequences that are the same or have a specified percentage of amino acid residues that are the same (e.g., at least 70%, at least 75%, at least 80%, 85%, 90%, 91%, 92%, 93%, 94%, 95%, 96%, 97%, 98%, 99%, or higher) identity over a specified region, e.g., the length of the two sequences, when compared and aligned for maximum correspondence over a comparison window or designated region. Alignment for purposes of determining percent amino acid sequence identity can be performed in various methods, including those using publicly available computer software. Examples of algorithms that are suitable for determining percent sequence identity and sequence similarity the BLAST 2.0 algorithms, which are described in Altschul et al., Nuc. Acids Res. 25:3389-3402 (1977) and Altschul et al., J. Mol. Biol. 215:403-410 (1990). Thus, for purposes of this invention, BLAST 2.0 can be used with the default parameters to determine percent sequence identity.

Sequence identity can be also be determined by inspection. For example, the sequence identity between sequence A and sequence B, aligned using the software above or manually (to maximize alignment), can be determined by dividing the length of sequence A, minus the number of gap residues in sequence A, minus the number of gap residues in sequence B, by the sum of the residue matches between sequence A and sequence B, times one hundred.

### Methods of Tuning Proximity Assays

Provided herein is a method of tuning a proximity reporter assay. The method includes providing a first series of compositions comprising first binding agents comprising a 5′ nucleic acid molecule and second binding agents attached to a 3′ nucleic acid molecule, wherein compositions in the first series comprise the first and second binding agents at different concentrations from the other compositions in the first series, and wherein the first and second binding agents bind a first target analyte. The methods also include providing a second series of compositions comprising third binding agents comprising a 5′ nucleic acid molecule and fourth binding agents comprising a 3′ nucleic acid molecule, wherein compositions in the second series comprise the third and fourth binding agents at different concentrations from the other compositions in the second series, and wherein the third and fourth binding agents bind a second target analyte.

The series of compositions can include any number of compositions. For example, the first series of compositions comprises 2, 3, 4, 5, 10, 15, 20, 25, 30, 35, 40, 45, 50 or more compositions. Optionally, the second series of compositions comprises 2, 3, 4, 5, 10, 15, 20, 25, 30, 35, 40, 45, 50 or more compositions.

The series of compositions are then used to form a plurality of mixtures. Thus, the methods can include forming a plurality of mixtures by contacting each composition from the first and second series of compositions with the first and second target analytes. The plurality of mixtures are then used in a proximity assay. Thus, the herein provided methods can include the step of performing, for each mixture in the plurality of mixtures, a proximity assay, wherein the assay produces, for each mixture, a signal comprising a nucleic acid product of the 5′ and 3′ nucleic acid molecules, wherein a first signal is produced for the first target and a second signal is produced for the second target.

The signals produced for the targets are used to select a single composition containing the binding agents at a particular concentration that results in signals within close range of magnitude in order to determine the concentration of the analytes in the sample. Thus, the methods include selecting from each of the first and second series of compositions a single composition comprising binding agents at a concentration that results in first and second signals within four orders of magnitude for the first and second target analytes. The first and second signals can be within three, two, or one orders of magnitude for the first and second target analytes. Optionally, each mixture comprises both of the first and second target analytes.

Once the compositions from the first and second series of compositions are selected an additional proximity assay can be used with the selected compositions. Thus, the methods can include performing a proximity assay using the selected compositions by contacting the selected compositions with a biological sample comprising the first and second target analytes and determining the concentrations of the first and second target analytes in the biological sample based on the assay.

As described herein the provided methods can be used to determine the concentration of analytes in a sample even if the analytes are present in the sample at very different concentration levels. For example, the first and second target analytes can be present in the mixtures at a first and second concentration, respectively, and wherein the first and second concentrations vary by at least three orders of magnitude. Optionally, the first and second concentrations vary by at least four, five, six, seven, eight, nine or ten orders of magnitude.

The target analyte can be present in the mixtures at any number of concentrations. The target analyte can be a low, medium or high abundance target analyte. Optionally, the second target analyte is a high abundance target analyte. Optionally, the first target analyte is a low or medium abundance target analyte. By way of example, high abundance targets can be present in a sample at a concentration from 100 picomolar (pM) to 10 millimolar (mM) or any concentration between 100 picomolar and 10 millimolar. A medium abundance target can be present in a sample at a concentration of 100 femtomolar (fM) to 100 picomolar or any concentration between 100 femtomolar and 100 picomolar. A low abundance target can be present fin a sample at a concentration less than 100 femtomolar, e.g., 100 attomolar (aM) to 100 femtomolar.

The binding agents can be, for example, antibodies, e.g., polyclonal antibodies. Optionally, the binding agents are aptamers or nanobodies. In the provided methods, combinations of binding agents can be used. Thus, a combination of antibodies, aptamers and nanobodies can be used.

Optionally, the compositions in the first or second series of compositions further comprises unlabeled binding agents, e.g., antibodies, aptamers, nanobodies, or combinations thereof, that bind the target analyte. The binding agents are unlabeled in the sense that they do not have a nucleic acid molecule that will take part in the proximity assay. Optionally, each composition in the first series of compositions further comprises unlabeled binding agents that bind the first target analytes. Optionally, each composition in the second series of compositions further comprises unlabeled binding agents that bind the second target analytes. Unlabeled binding agent concentrations in the compositions depends on target concentration. In other words, the greater the target concentration the greater the unlabeled binding agent concentration. For example, for a 100 picomolar target analyte concentration, the unlabeled binding agent concentration can be from 100 picomolar to 1 nanomolar in the composition. By way of another example, for a 1 nanomolar (nM) analyte, the unlabeled binding agent concentration can be from 1 to 10 nM in the composition. For a 10 nM analyte, the unlabeled binding agent concentration can be from 1 to 10 nM or from 10 to 100 nM in the composition. For a 100 nM analyte, the unlabeled binding agent concentration can be from 100 to 1000 nM in the composition.

The proximity assay can be, for example, a proximity ligation assay, and wherein the proximity ligation assay comprises ligating the 5′ and 3′ nucleic acids thereby producing the nucleic acid product comprising the 5′ and 3′ nucleic acids. Once ligated, the proximity assay can further include amplifying the nucleic acid products. The amplified nucleic acid products can be the signal or the amplified nucleic acid products can be further analyzed to produce the signal. For example, the provided methods can include sequencing the amplified nucleic acids products. Optionally, the amplified nucleic acid products comprise a barcode sequence, which can be a target analyte specific barcode. As described herein, the amplified nucleic acid products may also include a unique molecular identifier.

The herein provided proximity assay can be repeated any number of times. For example, one or two or more times. Further, the proximity assay can be performed for any number of analytes.

Thus, also provided is a method of providing a tuned proximity reporter assay for N number of analytes. The method includes providing an N series of compositions for N analytes, wherein each series of compositions corresponds to a different analyte, wherein each series comprises compositions comprising first binding agents comprising a 5′ nucleic acid molecule and second binding agents attached to a 3′ nucleic acid molecule for one analyte in the N sets of analytes, wherein the compositions in each series comprise the first and second binding agents at different concentrations from the other compositions in the series. The method also includes, for each N series of compositions, forming a plurality of mixtures by contacting each composition from the series of compositions with the analyte from the N analytes that corresponds to the series of compositions and performing, for each mixture in the plurality of mixtures, a proximity assay, wherein the assay produces, for each mixture, a signal comprising a nucleic acid product of the 5′ and 3′ nucleic acid molecules, wherein each signal corresponds to an analyte. The method includes selecting for each N series of compositions a single composition comprising binding agents at a concentration that results in signals within four orders of magnitude for each analyte in the N analytes, wherein the selected compositions from the N series of compositions provides a multitude of compositions each composition comprising the selected concentration of binding agents for each analyte within the N analytes, thereby providing a tuned proximity reporter assay. The number of analytes can be, for example, 1, 2, 3, 4, 5, 6, 7, 8, 9, 10, 15, 20, 25, 30, 35, 40, 45, 50, 75, 100, 200, 300, 500, 750, 1,000, 3,000, 10,000, 30,000, or 100,000.

In the provided methods the compositions can include capture binding agents designed to capture the target analytes of interest. The capture binding agents are designed to retain the target analytes before they are contacted with the binding agents labeled with the nucleic acids to be used in the proximity assay. Thus, the first compositions can include first capture binding agents that bind the first analytes and the second compositions can include second capture binding agents that bind the second analytes. The capture binding agents can be attached to a solid surface, e.g., a bead.

In methods and compositions described herein, a solid support may be a material to which the herein provided molecules, e.g., analytes and agents, can be attached and is amenable to at least one detection method. Possible solid supports include, but are not limited to, a polystyrene surface, a polypropylene surface, a gold surface, a glass surface, or a silicon wafer. Other possible materials for a solid support may be, e.g., glass and modified or functionalized glass, plastics (including acrylics, polystyrene and copolymers of styrene and other materials, polypropylene, polyethylene, polybutylene, polyurethanes, TEFLON®, and the like), nylon or nitrocellulose, resins, silica or silica-based materials including silicon and modified silicon, carbon, metals, inorganic glass, plastics, optical fiber bundles, and a variety of other polymers.

Solid supports used in methods and compositions of the disclosure may be fashioned into a variety of shapes. The solid support can be circular, such as a bead, e.g., magnetic bead or polystyrene bead. The solid support can be, for example, a gold or silver nanoparticle. The solid support may be substantially planar, such as plates (e.g., slides), microtiter plates, flow cells, coverslips, microchips, and the like. The solid support can be, for example, well plates, plastic surfaces, glass slides, packed bed columns, gold electrodes, or fiber optics. The surface of the solid support may be further modified to contain tiles, wells, trenches, grooves, depressions, or the like. In further embodiments, microspheres or beads may be placed in the wells, i.e., through covalent conjugation. Further, the solid support may be modified to contain chemical functional groups, e.g., amino groups, carboxy groups, oxo groups, thiol groups, and the like.

As used herein, “bead” may refer to (but is not limited to) beads of the type used in droplet-based single cell sequencing technologies (inDrop, Drop-seq, and 10× Genomics) which carry or are attached to polynucleotides. Bead technology is well known in the art. Wang et al., 2020, “Dissolvable Polyacrylamide Beads for High-Throughput Droplet DNA Barcoding” Advanced Science 7:8, and references cited therein; Klein et al. Cell 2015, 161, 1187; Macosko et al., Cell 2015, 161, 1202; Lan et al Nat. Biotechnol. 2017, 35, 640; Lareau et al. Nat. Biotechnol. 2019, 37, 916; Stoeckius et al. Nat. Methods 2017, 14, 865; Peterson et al. Nat. Biotechnol. 2017, 35, 936; Zheng et al., Nat. Commun. 2017, 8, 14049.

As described herein, the methods include detecting a signal comprising a nucleic acid product. Nucleic acids can be detected in any number of known ways including PCR and sequencing. Methods for detecting nucleic acids include, for example, Northern blots, RT-PCR, arrays including microarrays and sequencing including high-throughput sequencing methods. If the nucleic acids include RNA, a reverse transcriptase reaction can be carried out and the targeted sequence is then amplified using standard PCR. Quantitative PCR (qPCR) or real time PCR (RT-PCR) is useful for determining relative expression levels, when compared to a control. Quantitative PCR techniques and platforms are known in the art, and commercially available (see, e.g., the qPCR Symposium website, available at qpersymposium.com). Nucleic acid arrays are also useful for detecting nucleic acid expression. Customizable arrays are available from, e.g., Affymatrix. Optionally, methods for detecting nucleic acids include sequencing methods. Sequencing methods are known and can be performed with a variety of platforms including, but not limited to, platforms provided by Illumina, Inc., (La Jolla, CA) or Life Technologies (Carlsbad, CA). See, e.g., Wang, et al., Nat Rev Genet. 10 (1): 57-63 (2009); and Martin, Nat Rev Genet. 12 (10): 671-82 (2011).

### EXAMPLES

### Example 1. A Multiplexed Detection Assay for Quantifying Proteins at Widely Divergent Concentrations

Blood-based quantification of protein biomarkers is a standard tool for the prediction, diagnosis, and monitoring of disease. Physiological concentrations of different plasma proteins can vary considerably, spanning over 10 orders of magnitude. There is currently no single assay that can quantify both low- and high-abundance proteins simultaneously from a single sample. Current methods rely upon sample splitting and differential dilutions—a strategy that can produce misleading results due to an effect known as non-linear dilution. Our ‘EVROS’ assay overcomes this problem with a two-pronged tuning strategy that makes it possible to normalize the signal output generated by analytes of widely divergent concentrations. The EVROS strategy was applied to a proximity ligation assay format and demonstrates the ability to simultaneously quantify four different proteins with physiological concentrations ranging from low femtomolar to high nanomolar-achieving an unprecedented dynamic range spanning seven orders of magnitude in a single 5 μL sample of undiluted serum. This capacity to accurately quantify such disparate analyte concentrations from tiny blood samples in a highly multiplexed fashion could be broadly used for biomarker discovery, disease monitoring, and other applications related to personalized medicine.

### Materials and Methods

### Reagents

All polyclonal antibodies were purchased affinity-purified from R&D Systems. Biotinylated and unbiotinylated antibodies were purchased for CRP (BAF1707 & AF1707), IL-6 (BAF206 & AF206), IL-1B (BAF201 & AF201), and IL-1RA (BAF280 & AF280). Unbiotinylated antibodies were purchased for GDF-15 (AF957) and GFP (AF4240). The GDF-15 and GFP antibodies were biotinylated using an EZ-Link Micro NHS-PEG4-Biotinylation Kit from Thermo Fisher Scientific (21955) according to the manufacturer's instructions. All recombinant target proteins except GFP were purchased from R&D Systems: CRP (1707-CR), IL-6 (206-IL), IL-1β (201-LB), IL-1RA (280-RA), and GDF-15 (957-GD). Recombinant GFP was purchased from Vector Laboratories (MB-0752). All oligonucleotides that were conjugated to antibodies, as well as the ligation splint, were purchased from Integrated DNA Technologies and were HPLC-purified. Oligonucleotide sequences are listed in Table 5. Purified DNA-conjugated antibodies were quantified via the Bradford assay, using the Bio-Rad Protein Assay Dye Reagent Concentrate (5000006) with 2 mg/mL Pierce bovine gamma globulin standard ampules from Thermo Fisher Scientific (23212) as a standard. Dynabeads MyOne Streptavidin T1 (65601), 50 mM D-biotin (B20656), UltraPure salmon sperm DNA solution (15632011), UltraPure 0.5 M EDTA, pH 8.0 (15575020), nuclease-free water (not DEPC-treated) (AM9932), SYBR Green I nucleic acid gel stain (10,000× concentrate in DMSO) (S7563), and the Qubit dsDNA HS Assay Kit (Q32851) were purchased from Invitrogen. 10× phosphate-buffered saline (PBS) solution, Tween 20, and molecular-biology grade 200-proof ethanol were purchased from Fisher BioReagents (BP399 & BP337). Molecular biology-grade bovine serum albumin (BSA) was purchased from New England BioLabs (B9000S). Goat IgG was purchased from Millipore Sigma (15256). Ampligase DNA Ligase and 10× Reaction Buffer were purchased from Lucigen (A3202K). 2×GoTaq G2 Hot Start Colorless Master Mix was purchased from Promega (9IM743). Uracil-DNA glycosylase (UDG) (1 U/μL) was purchased from Thermo Fisher Scientific (EN0362). Nextera XT Index Kits (96 indexes, 384 samples) were purchased from Illumina (FC-131-1002). Axygen AxyPrep Mag PCR Clean-up Kits were purchased from Thermo Fisher Scientific (MAGPCRCL). Buffer EB was purchased from Qiagen (19086). DynaMag-96 side magnets were purchased from Thermo Fisher Scientific (12331D). Viaflo 96-channel pipette and 300 μl pipette tips were purchased from Integra BioSciences (6432). All gel electrophoresis reagents were bought from Thermo Fisher Scientific: Novex 10% TBE (EC6875BOX), 6× loading dye (R0611), and O'RangeRuler 20 bp (SM1323). DNA quantification is done by Quant-iT ds DNA HS Assay (Q33120) via Thermo Fisher Scientific. Human serum samples were obtained from BioIVT (HUMANSRMUNN): male human serum, lot numbers HMN613433, HMN613434, and HMN613436, aged 38, 38, and 45 years old, respectively. Luminex reagents were purchased from EMD Millipore Corporation, Burlington, MA.

### Detection Probe Functionalization

The detection probes were generated by conjugating amine-modified oligonucleotides to polyclonal antibodies using the Antibody-Oligonucleotide All-in-One Conjugation Kit by TriLink Biotechnologies (A-9202). Oligos attached at the 3′ end also featured 5′ phosphorylation. Conjugations were performed according to the manufacturer's instructions, with the exception that the antibody/DNA attachment reactions were run overnight at 4° C. rather than for the recommended two hours at room temperature, as the oligonucleotides were longer (80-81 nucleotides) than the suggested maximum of 60 nucleotides recommended by the kit manufacturer.

### Solid-Phase Proximity Ligation Assay

The SP-PLA portion of the assay was run with only minor modifications from published protocols^{17,26}. Capture antibody (cAb)-coated beads were prepared with MyOne Streptavidin T1 beads following the manufacturer's protocol. The stock beads (10 mg/ml) were resuspended on a rotator for 5 min. For each target, 50 μL beads were added to a 1.5-mL microcentrifuge tube. The tubes were placed on a magnet to pellet the beads, the storage buffer was removed, and the beads were washed three times with 200 μL wash buffer (1×PBS+0.05% Tween 20). Biotinylated antibodies were reconstituted in storage buffer (1×PBS+0.1% BSA) at 50 nM for all targets except CRP, which was reconstituted at 1.33 μM. After the third bead wash, 100 μL of biotinylated antibody solution was added to each tube. The beads were briefly vortexed to homogenize the solution, then incubated for 1 h at room temperature on a rotator. The tubes were then placed on a magnet, the supernatant was removed, and the beads were washed three times with 200 μL wash buffer. The beads were then resuspended in 100 μL storage buffer and used immediately or kept at 4° C. until used. CRP cAb beads were prepared using 2.5-fold greater volumes than indicated above, with the indicated higher antibody concentration for maximum binding capacity to reduce capture antibody saturation.

PLA buffer (1×PBS, 1 mg/mL BSA, 0.05% Tween 20, 15 μg/m goat IgG, 0.1 mg/mL salmon sperm DNA, and 5 mM EDTA) was prepared with GFP spiked in so that its final concentration in every sample would be 10 pM. GFP was used as an internal control to monitor variability in PLA steps, including ligation efficiency, bead washing, etc., with an exogenous analyte not found in human serum. Standard curves in GFP-spiked buffer were prepared by 3× serial dilution of a tube containing a mix of every target at its highest standard curve concentration. Non-target-spiked samples were prepared by simply using GFP-spiked buffer. For each sample, 40 μl of PLA buffer is added to a well in a 96-well plate.

Separately, the previously prepared cAb-coated beads (5 mg/mL) were homogenized via vortexing, and a 50 μL reaction was prepared in a tube containing 167 nL of 5 mg/mL bead solution (˜700,000 beads) for each target. To avoid bead saturation, CRP required 10 times more beads than the other targets, or ˜7,000,000 beads per 50 μL reaction. After adding each species of cAb-coated bead to a single tube, the tube was placed on a magnet, the storage buffer was removed, and the beads were resuspended in GFP-spiked PLA buffer to produce a solution in which each cAb-coupled bead species was present at 167 ng/ml (1.67 μg/mL for CRP). After mixing via vortexing, 5 μL of this bead solution was added to each 40 μL sample in the PCR plate. Finally, 5 μl of buffer or undiluted chicken or human serum were added to each well. All samples were run in triplicate, with each sample having a final volume (after reagent addition) of 50 μL, and final GFP concentration of 10 pM. After sealing the plate, the samples were gently vortexed for 1 min and incubated on a rotator for 1.5 h at room temperature.

Following this incubation, the plate was spun down at 1000 rcf for 5 s and placed on a DynaMag 96-Well plate for 1 min to pellet the beads. The supernatant was removed, and 100 μL wash buffer added to each well using custom protocols on a Viaflo 96 channel pipette. The plate was again sealed, gently vortexed for 1 min, spun down at 1000 rcf for 5 s, and placed on a magnet. The wash buffer was removed, and another wash performed as described above. Following the removal of the wash buffer from the second wash, each well was filled with 50 μL of PLA probe solution in non-GFP-spiked PLA buffer, containing each target's probes at their optimized concentrations, as well as any unlabeled polyclonal antibodies used as an epitope depletant. The plate was sealed, vortexed gently for 1 min, and incubated for 1.5 h on a rotator at room temperature.

The plate was again spun down and placed on the magnet. The supernatant from each well was removed with the Viaflo, and the wells were washed two times with 100 μL wash buffer as described previously. Following the second wash, each well was filled with 50 μL of ligation reaction solution, consisting of 0.05 U/μL Ampligase, 100 nM ligation splint, 1× Ampligase reaction buffer, and nuclease-free water. The plate was again sealed, gently vortexed for 1 min, spun down at 1000 rcf for 30 s, and placed in a thermocycler and incubated at 50° C. for 10 min to allow the ligation reaction to occur. Following this reaction, the plate was spun down at 1000 rcf for 1 min and placed on a magnet. The supernatant was removed, and the wells were again washed twice with 100 μL wash buffer.

Following the second wash, 40 μL of PCR reaction solution was added to each well, consisting of 1× GoTaq G2 Hot Start Colorless Master Mix, 0.02 U/μl UDG, 100 aM control oligo, and PCR-grade water. Samples were placed at 4° C. overnight to continue the protocol the next day. To each sample, 5 μL of each of two different Nextera XT indices (10× stock) was added. These indexes act both as PCR primers to amplify the DNA as well as sample indices to uniquely identify each sample, as the pair of indices added to each well in a PLA-Seq run will be unique to that run. This enables the pooling of all the samples from a single PLA-Seq run (up to 384 samples with the original index kit, or higher if combining index kits). Following sequencing, these are de-multiplexed based on their unique index pair.

Following the addition of the indices, the plate was sealed, gently vortexed, spun down for 1 min at 1000 rcf, and placed in a thermocycler for a preamplification reaction: 10 min at 95° C. (to activate the polymerase and deactivate the UDG), 72° C. for 3 min, 95° C. for 30 s, and 4 cycles of 95° C. for 10 s, 55° C. for 30 s, and 72° C. for 90 s, followed by 5 min at 72° C. and 5 min at 4° C. The plate was then spun down at 1000 rcf for 1 min and placed on a magnet. From each 50 μL reaction, 33 μL was removed and transferred to a new PCR tube, which was stored at 4° C. To the remaining 17 μL/well, 2 μL 10×SYBR Green I dye was added. The plate was covered, gently vortexed for 1 min, and spun down for 1 min at 1000 rcf. The plate was then analyzed via qPCR to determine the number of cycles that each sample (i.e., the 33 μL in the PCR tubes) should be amplified so that all samples produce roughly the same amount of total DNA. The qPCR protocol was: 72° C. for 3 min, 95° C. for 30 s, and 39 cycles of 95° C. for 10 s, 55° C. for 30 s, and 72° C. for 30 s. To ensure that every sample is amplified to roughly the same DNA output, the number of cycles required was calculated for each sample to reach 0.25 maximum fluorescent value (Ct).

Ct values were extracted with a custom python script. Each amplification intensity value (Iᵢ) across all 39 cycles was normalized by:

$${I}_{i \_ norm} = \frac{{I}_{i} - min ( I )}{max ( I ) - min ( I )} \qquad [ Equation 1 ]$$

Using these normalized values, the cycle that first passes the threshold value of 0.25 was the Ct value.

This value was then used as the number of amplification cycles for the remaining 33 μL of sample. If samples varied by <3 cycles, all samples were amplified to 1+average Ct. The amplification protocol itself was identical to the qPCR protocol (without plate reads); after amplification was complete, samples were removed during the 90 s 72° C. step and incubated in another thermocycler at 72° C. for an additional 5 min before being removed and placed at room temperature. Once every sample was amplified, PCR cleanup was performed using an Axygen Axy Prep Mag PCR Clean-up Kit based on the manufacturer's instructions, using the Viaflo for high throughput. The resulting purified PCR products were then quantified using the Quant-iT dsDNA protocol. Samples were then pooled together at equal molar concentrations. The samples were quality-controlled by native gel electrophoresis at 180V for 40 min. The pool was then quantified again with Qubit and sent for sequencing on an Illumina MiSeq at the Stanford Functional Genomics Facility.

For a qPCR readout (as in the tuning experiments), normal PLA buffer was used throughout the above protocol instead of GFP-spiked PLA buffer. Additionally, after the wash following ligation, 50 μl of the following PCR mix was added to the beads: 1×GoTaq G2 Hot Start Colorless Master Mix, 0.02 U/μl UDG, 0.25×SYBR Green, 500 nM Universal Forward Primer, 500 nM Universal Reverse Primer, and PCR-grade water. For the qPCR readout, the protocol above ended with the Cq extraction.

DNA Calibration Curves for qPCR:

To convert qPCR Ct values to approximate DNA concentration, a calibration curve with a serial dilution of full sequence of the IL-1ra reporter was made. qPCR measurements were conducted in triplicate for 100, 10, 1, 0.1, 0.01, and 0 fM DNA, and calculated Ct values. Since the Ct values were highly linear with log (input DNA template), the following equation was fitted

$$Ct = m * log ( [ DNA ] ) + b \qquad [ Equation 2 ]$$

where Ct is the calculated 0.25 max value obtained from qPCR amplification of the input template DNA concentration. The parameters m and b were kept for all future conversions from Cq to [DNA] as follows:

$$[ DNA ] = 1 {0}^{\frac{{C}_{q - b}}{m}} \qquad [ Equation 3 ]$$

### Data Extraction Analysis:

FASTQ files were analyzed using custom code written in Perl. Code is available at https://github.com/brandonwilsonphd/EVROS. After demultiplexing in BaseSpace according to Illumina indices, reads were filtered by quality; only reads 52 nt in length with Phred scores ≥20 for all bases were used in our analysis. Duplicate reads were compiled and counted using the fastaptamer_count package²⁷. After each UMI was only represented once per file, the compiled reads were aligned to the known reporter sequences (Table 4). An exact similarity score was used, as opposed to Needleman Wunsch or SmithWaterman, because the split regions contain high degrees of similarity shifted by a few bases. An exact similarity score minimizes false positives by emphasizing the sequence similarity in the protein tag region. Each read was assigned to the reporter with the highest similarity score, provided the similarity score was greater than 31. Reads that returned similarity scores <31 for all reporters were discarded. The resulting output comprises the total number of unique barcodes for every target reporter.

Each sample also included an internal control reporter oligo to reduce intra-assay variance that arises from variations in experimental factors such as ligation efficiency, library prep, and pooling for sequencing. Outputs were reported in terms of normalized unique molecular identifiers (nUMI), which reflects the number of UMIs for the target divided by the number of UMIs for the control reporter oligo.

### Curve Fitting and Quantification:

Curve fitting and quantification were done with a custom python script. To create a calibration curve, the data was fitted to the Four-Parameter Logistic Curve (4-PL),

$$nUMI = \frac{A - D}{1 + {( \frac{x}{C} )}^{B} ]} + D \qquad [ Equation 4 ]$$

where nUMI is the normalized counts from sequencing, x is the analyte concentration, A is the minimum value possible with no analyte, B is the Hill coefficient, C is the point of inflection (K_{d}), and D is the maximum value possible with infinite analyte. The parameters A, B, C, and D were determined by the curve-fitting function in python using Scipy's optimize curve fit function, which uses non-linear least squares to fit a function. The log residual was used for the loss function. To calculate the concentration for a given signal, the inverse function was used with fit parameters A, B, C, D as follows:

$$x = C {( \frac{A - D}{nUMI - D} - 1 )}^{1 / B} \qquad [ Equation 5 ]$$

### Luminex Sample Prep:

Three separate Luminex panels were used for the four targets according to the manufacturer's protocols: Human Cardiovascular 2 MAG (GDF-15; Cat #MXHCV2M0N02189), Neurodegenerative MAG Panel 2 (CRP; Cat #MXHNDG2M0N02068), and Human Cyto/Chem/GF Panel A (IL-6, IL1-RA; Cat #MXHCYTA0N03031). The recommended sample dilutions for each panel (3×, 100×, and 1000×, respectively) were prepared with 1×PBS. Kit-provided standards were prepared with four-fold serial dilutions with assay buffer. For each sample, 25 μl of buffer or matrix (per manufacturer's recommendation—buffer for CRP and GDF-15 panels, and matrix for IL-1ra/IL-6 panels) was added with 25 μl of magnetic beads and 25 μl of sample at the recommended dilution or standard. Samples were incubated for 16 h at 4° C. on a shaker. After incubation, the samples were washed in a magnetic washer and 25 μl of detection was added and incubated for 1 h. SAPE was added for 30 min at room temperature on a shaker, and the samples were then washed on a magnetic washer. After adding 130 μl of reading or wash buffer, the samples were placed on a shaker for 3 min, then placed in a Luminex 100/200 System for reading. All samples were tested in duplicate and analyzed with standard quantification procedures recommended by Luminex.

The dilutions spanned a three-log range: neat (no dilution), 3-fold, 10-fold, 100-fold, 1,000-fold, and 2,000-fold. Serum samples were diluted in PBS in a master plate and applied to all four panels. All sample dilutions were analyzed on all three panels. 25 μl of the diluted sample was mixed with antibody-linked magnetic beads in a 96-well plate and incubated overnight at 4° C. with shaking. Cold and room temperature incubation steps were performed on an orbital shaker at 500-600 rpm. Plates were washed twice with wash buffer in a BioTek ELx405 washer. Following 1 h incubation at room temperature with biotinylated detection antibody, streptavidin-PE was added for 30 min with shaking. Plates were washed as described above and PBS added to wells for reading in the Luminex FlexMap3D Instrument with a lower bound of 50 beads per sample per cytokine. Each sample was measured with two replicates. Wells with a bead count <50 were flagged, and data with a bead count <20 were excluded.

### Results and Discussion

### Assay Overview

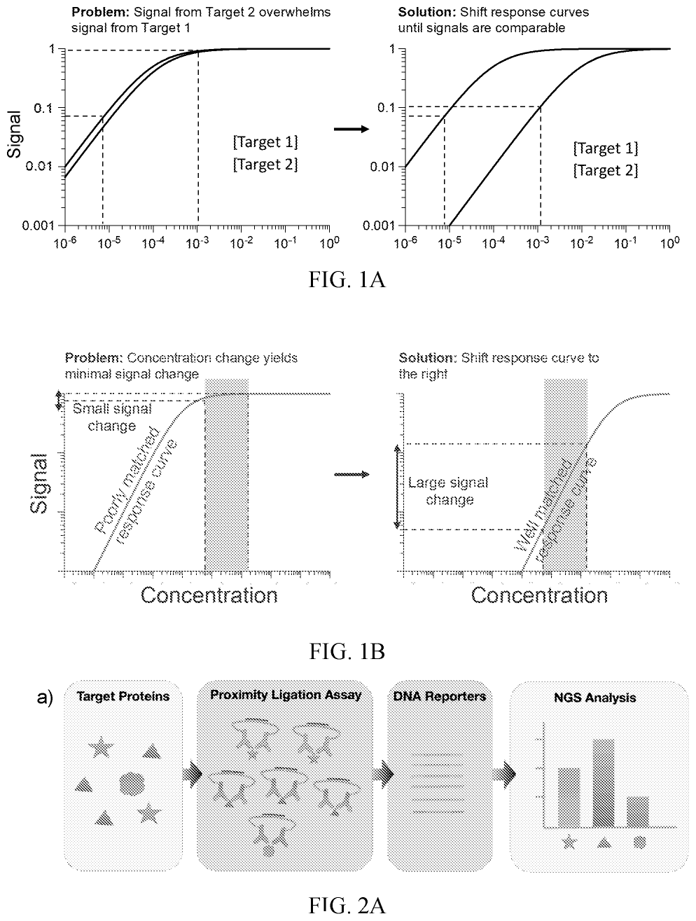

Two factors limit the dynamic range of any multi-analyte detection system. First, the signals from high-abundance analytes can overwhelm the signals from low-abundance analytes if the analytes have similar response curves (FIG. 1A, top). Second, detection ranges are generally limited by the binding affinities of the detection reagents used in an assay. More specifically, the concentrations of high-abundance analytes can greatly exceed the affinity of their corresponding detection antibody, such that changes in physiological concentration are likely to occur in the saturated plateau of the signal response curve, and thus result in only small changes in signal (FIG. 1B, top). Together, these limitations make it difficult—if not impossible—to quantify high- and low-abundance analytes simultaneously from the same sample. However, if the response curve for every analyte can be modulated independently, these limitations can be overcome by ensuring that both (i) high- and low-abundance targets produce signal outputs that are on the same order of magnitude (FIG. 1A, bottom) and (ii) that the signal response is appropriately sensitive to every target within the relevant concentration range (FIG. 1B, bottom).

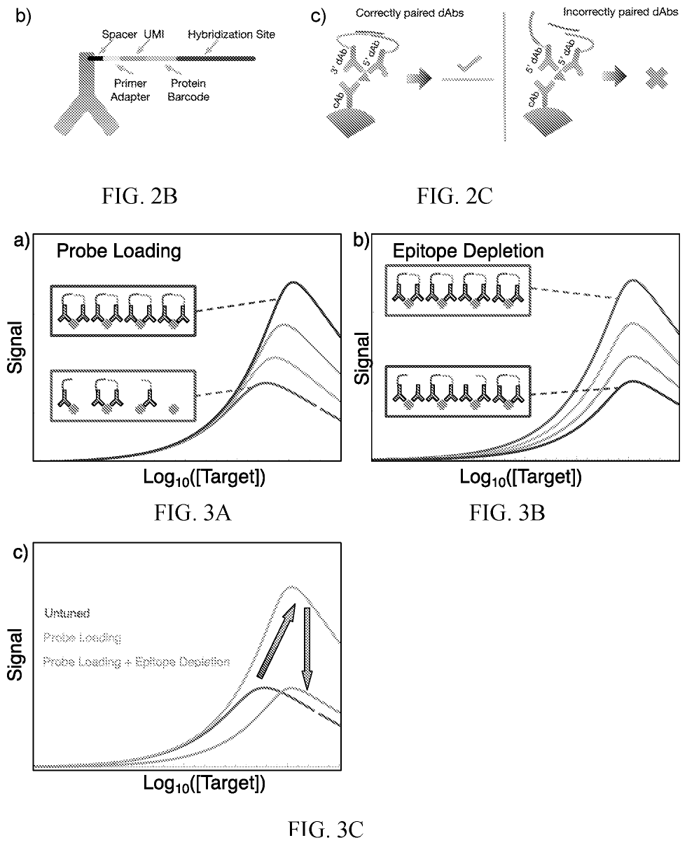

EVROS introduces two independent tuning mechanisms to modulate the signal response curve of each analyte individually. First, our ‘probe loading’ strategy ensures that similar signals are produced for all targets regardless of their abundance. Second, our ‘epitope depletion’ strategy shifts the binding curve of our detection reagents to match the physiological concentration range of the target. In principle, these two tuning mechanisms can be applied to many types of immunoassays, but EVROS has been exemplified in the context of spPLA. This assay format employs polyclonal antibodies that are divided into three pools for each target—one of which is coupled to magnetic beads and acts as a capture antibody, and two pools of DNA-labeled dAbs. This approach eliminates the need to screen multiple sets of monoclonal antibodies, because polyclonal antibody pools are very likely to bind multiple distinct epitopes on a target simultaneously^{9,18} When the two dAbs simultaneously bind to the same captured target molecule, their associated DNA strands can undergo a ligation reaction in the presence of a complementary ‘hybridization splint’ DNA strand (FIG. 8), generating target-specific DNA reporter sequences that are subsequently amplified and sequenced (FIG. 2A). These reporters contain protein-specific tags and unique molecular identifiers (UMIs)¹⁹ that enable us to assign each target an individual read count based on the sequencing data. One set of dAbs for each target is functionalized with a DNA strand attached by its 5′ end, while the other dAb is coupled to its DNA strand by the 3′ end (FIG. 2B), such that only target molecules bound by correctly paired 5′- and 3′-dAbs will generate a DNA reporter (FIG. 2C).

### Independently Tuning the Signal Output for Each Analyte

Our probe-loading tuning mechanism entails changing the concentrations of the dAbs to ensure that all analytes produce similar output signals, even if their original concentrations differ by many orders of magnitude (FIG. 1A). This prevents any analyte from overwhelming the signal from another, lower-abundance analyte. Probe loading shifts the signal response curve as a result of the change in equilibrium based on Le Chatelier's Principle: as the total concentration of dAbs ([A]ₜₒₜ) increases, the equilibrium shifts towards target molecules that are bound to a greater number of dAbs (FIG. 3A). The equilibrium model of binding between dAbs and targets is described in detail in Supplemental Note 1 and forms the conceptual framework through which the two tuning mechanisms were analyzed.

The epitope depletion tuning mechanism modulates the DNA reporter output for an analyte by controlling the fraction of dAb pairs that result in a signal (FIG. 3B). This was achieved through the addition of unlabeled ‘depletant’ antibodies, which are derived from the same original polyclonal pool as the dAbs, but without attaching DNA oligos. The addition of depletant decreases the probability that a target bound to multiple antibodies will produce a signal according to the binomial distribution (Supplemental Note 1). The efficiency with which DNA reporters are generated can be modulated by tuning the fraction of depletant antibody, which results in a downward shift of the response curve. Epitope depletion by itself results in a much lower output signal, so this measure was combined with increased probe loading to obtain a readout that is of the same order of magnitude as the other targets.

By using both tuning mechanisms together, the response curve can be shifted to the right, providing greater resolution for high-abundance analytes by shifting the inflection point of the effective response curve to match their concentration range, while also balancing the magnitude of the signal output (FIG. 3C). Without such tuning, the signal from these high-abundance analytes would be saturated and yield minimal change in signal in response to shifts in analyte concentration as shown in FIG. 1B.

### Experimental Demonstration of the Two Tuning Mechanisms

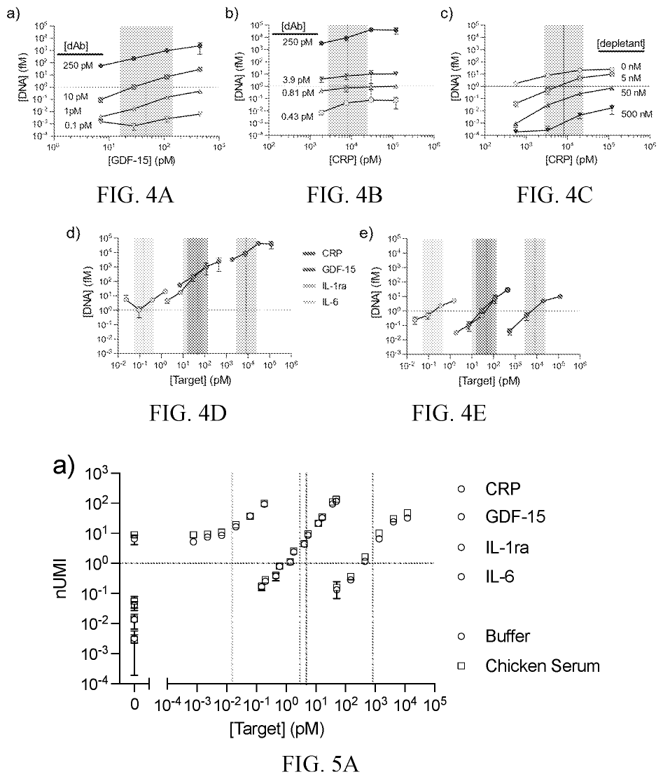

The ability to predictably shift analyte response curves was demonstrated with the growth/differentiation factor-15 (GDF-15) protein, which is a biomarker for inflammation, myocardial ischemia, and cancer. Since physiological ranges can vary as a function of disease state, the testing range (5-500 pM) was designed to be broader than the nominal concentration range of ˜5-120 pM (0.2-5.0 ng/mL) in serum. The goal was to shift the response curve until the DNA reporter output concentration was ˜1 fM at the log-middle of the tested concentration range (˜50 pM). This target output was established empirically as the concentration of DNA reporter that maximizes the quantitative precision conferred by the UMIs (FIG. 9). To expedite the tuning process, the DNA reporter output was measured with quantitative PCR (qPCR) rather than the full sequencing workflow. Starting from standard spPLA conditions of 250 pM of each dAb, the assay was tuned in buffer by incrementally decreasing the probe concentration. As expected, decreasing [A]ₜₒₜ reduced the reporter output of the system (FIG. 4A), shifting the response curve downwards—equivalent to shifting the linear response curve to the right. Ten (10) pM was identified as the optimal concentration for each dAb, yielding an output of ˜1 fM DNA at the center of the tested concentration range of GDF-15. This trial-and-error tuning process is relatively straightforward but would be tedious and time-consuming to perform at scale for hundreds or even dozens of targets. For this reason, heuristics was developed based on empirical trends from past tuning data to rapidly optimize probe concentrations (Supplemental Note 2).

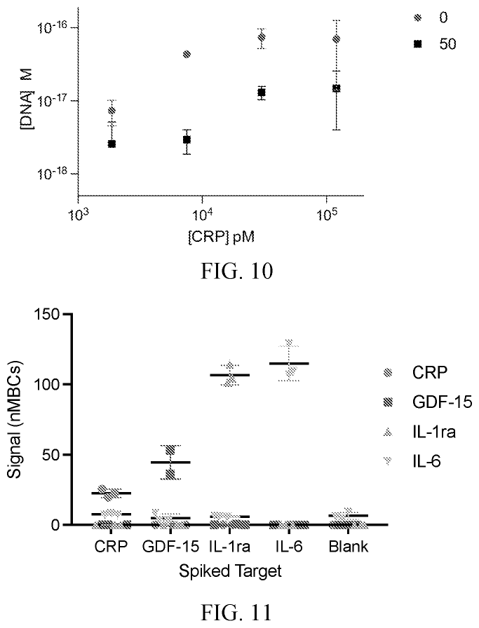

For high-abundance proteins, it is difficult to accurately quantify changes in concentration that occur in the saturated, plateau region of the signal response curve (FIG. 1B). The goal here is therefore to shift the curve to the right, to generate a more sensitive signal response over the desired concentration range within the steep-sloped portion of the curve. Epitope depletion makes this possible—but since epitope depletion by itself would result in a very low output signal, this measure was combined with increased probe loading to obtain a readout that is of the same order of magnitude as the other targets. It was demonstrated this with CRP, a high-abundance target with a basal concentration range of 1-36 nM. At the initial dAb concentration of 250 pM, the reporter DNA output concentration far exceeded our target of 1 fM at the log-middle of the CRP testing range (1-70 nM). Our tuning heuristic correctly predicted that decreasing the dAb concentration to 0.81 pM would commensurately decrease reporter DNA output to 1 fM (FIG. 4B). However, this probe tuning step alone was insufficient to achieve quantitative resolution of CRP over the desired concentration range and produced a very flat response curve. Epitope depletion and probe loading was applied together to steepen the signal-response curve while also shifting it to the right. It was determined that adding 50 nM of depletant would result in a roughly 10-15-fold drop in DNA output signal (FIG. 10), and therefore used our heuristic to identify an initial probe loading concentration at which the predicted reporter output would be 10-fold higher: 10 fM rather than 1 fM. It was determined that a 3.93 pM concentration of each dAb would appropriately compensate for the reduced reporter output due to epitope depletion (FIG. 4B). After testing a range of depletant antibody concentrations (FIG. 4C), it was determined that 5 nM depletant produced the optimal signal response, demonstrating that the use of both tuning mechanisms in tandem makes it possible to generate a highly sensitive response to high-abundance analytes in buffer. It should be noted that the detection of high-abundance analytes in proximity-based assays is often plagued by the “hook effect”, in which high target concentrations lead to decreased signal; this phenomenon and how it was evaded is discussed below in Supplemental Note 3.

### Four-Plex Detection of Analytes at Highly Divergent Concentrations in Undiluted Serum

The tuning mechanisms described above make it possible for us to achieve quantitative detection of multiple targets with concentrations spanning a vast dynamic range in a single measurement. As a demonstration, a four-plex EVROS assay was performed to simultaneously measure CRP, GDF-15, interleukin-1 receptor agonist (IL-1ra), and IL-6 spiked into buffer and serum at physiologically relevant concentrations. The basal physiological ranges of these proteins collectively span seven orders of magnitude (Table 2). In an untuned sp-PLA assay with a qPCR readout, it was found that the DNA output concentrations from these four targets spanned more than five orders of magnitude across the tested target concentrations, with especially poor resolution for high-abundance analytes (FIG. 4D). Consequently, the read-based signal from CRP would drown out that from IL-6 in an untuned assay.

Using our tuning heuristics, the optimal probe and depletant concentrations that would produce reporter DNA output for all targets within a range spanning three orders of magnitude was determined (FIG. 4E, Table 3). This tuning process conferred the capability to measure all targets in the desired concentration range simultaneously using a sequencing-based readout. To demonstrate this, six standards were prepared containing different concentrations of the four analytes (Table 1). These were designed to simulate the analyte concentrations that might be observed in human serum, and all experiments were conducted with 50 μL reactions that consisted of 5 μL of buffer plus 45 μL of assay reagents (dAbs, hybridization splint, standard containing spiked targets, etc.).

| Standard | [CRP] pM | [GDF-15] pM | [IL-1RA] pM | [IL-6] pM |
|---|---|---|---|---|
| 1 | 121500 | 486 | 364.5 | 1.823 |
| 2 | 40500 | 162 | 121.5 | 0.608 |
| 3 | 13500 | 54 | 40.5 | 0.204 |
| 4 | 4500 | 18 | 13.5 | 0.068 |
| 5 | 1500 | 6 | 4.5 | 0.023 |
| 6 | 500 | 2 | 1.5 | 0.008 |

In the multiplexed assay, an NGS readout was employed rather than qPCR. Instead of detecting DNA reporter concentration, nUMI—the number of UMIs associated with an analyte normalized by the number of control oligo UMIs was reported. This normalization accounts for variabilities such as differences in library pooling. EVROS consistently produced quantitative binding curves for all four analytes in assays performed with all six standards in buffer (FIG. 5A, circles). As expected, the signal outputs for all analytes were concentrated within a range spanning three orders of magnitude, such that none of the analytes' signals drowned out those from the other analytes. As such, low femtomolar concentrations of IL-6 and high nanomolar concentrations of CRP in a single sample were able to be measured. Importantly, EVROS exhibited excellent target specificity, as predicted for an spPLA-based assay: when individual analytes were spiked into reactions containing detection reagents for all four analytes, the assay consistently generated an appropriate analyte-specific readout with no off-target signal (FIG. 11). With the current targets, it was demonstrated detection of protein concentrations spanning over seven orders of magnitude, from 8 fM (IL-6) to 122 nM (CRP) (Table 1).

Complex biological matrices such as serum contain abundant interferents that can greatly impair the performance of immunoassays relative to results obtained in buffer. It was therefore tested whether EVROS can still obtain high resolution measurements in undiluted chicken serum. This medium was chosen because antibodies against human proteins typically do not cross-react with their chicken homologues, thereby minimizing the confounding effects of endogenous proteins in the serum sample⁸. Using the same optimized reaction conditions, the same six standards were measured as described above—with 5 μL undiluted chicken serum instead of buffer—to produce binding curves for all four analytes (FIG. 5A, squares). It was observed highly consistent normalized UMI (nUMI) values for each analyte in both buffer and chicken serum (linear fit: m=1.004±0.011, b=0.070±0.011 nUMI, R²=0.992), indicating the assay's robustness in complex sample matrices (FIG. 5B). The slope of 1 and y-intercept of 0.07 further indicate a good match between the nUMI counts generated under different reaction conditions.

### Four-Plex Detection of Analytes in Undiluted Human Serum

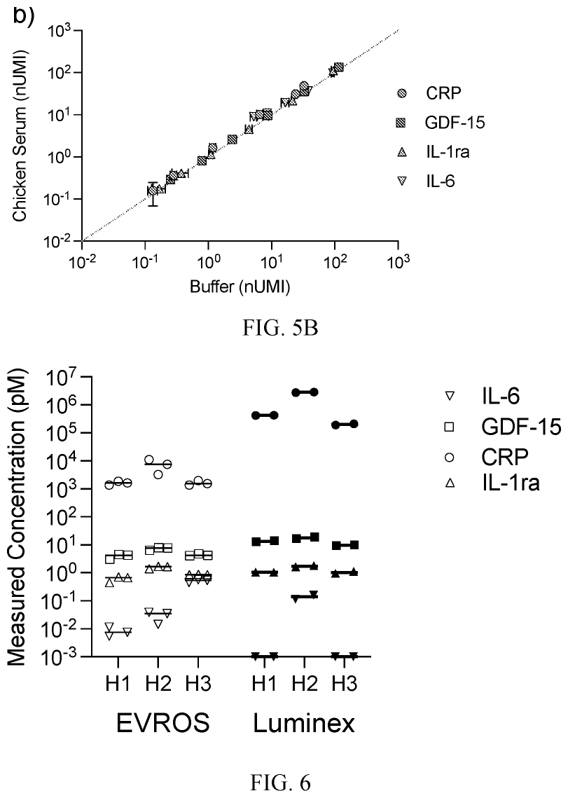

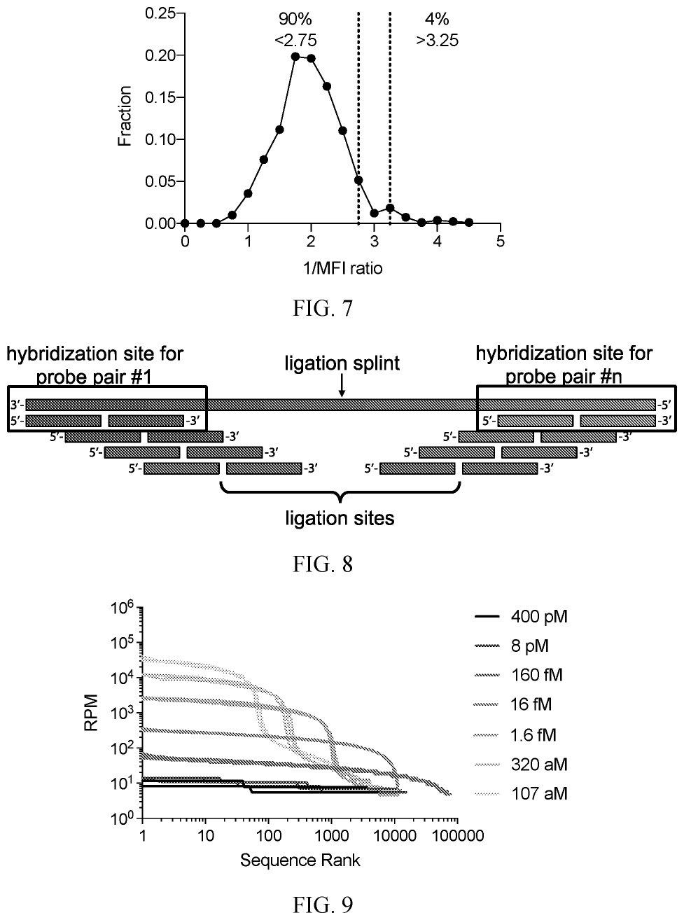

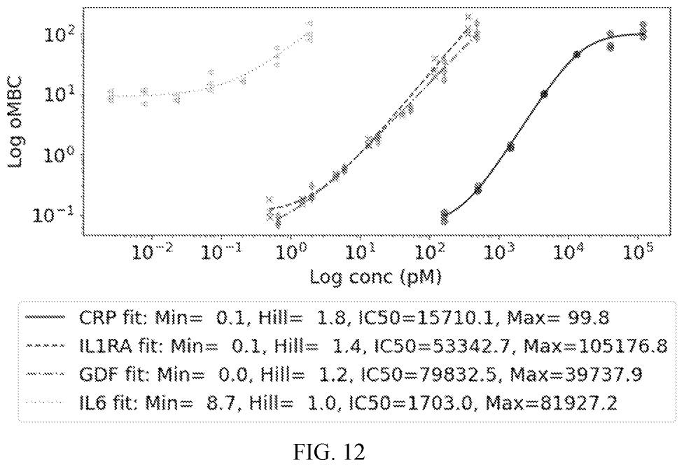

To demonstrate the real-world utility of our assay, endogenous concentrations of IL-6, IL-1ra, GDF-15, and CRP were measured in undiluted human serum samples. The reaction conditions were replicated above, using 5 μl of serum samples from three anonymous donors (see Methods) combined with 45 μl of EVROS assay reagents, with no spiked-in target protein. After sequencing and obtaining nUMI counts for each target in sample, their respective concentrations were calculated using binding curves developed with the six standards in buffer (see Methods, FIG. 12). IL-6, IL-1ra, GDF-15, and CRP were directly detected and quantified simultaneously in all three samples (FIG. 6, left). The measured endogenous target concentrations for all four analytes were consistent with previously reported physiological values—but notably, not identical across donors—in all three samples. This represents the first time such a broad range of endogenous analyte concentrations, ranging from low femtomolar IL-6 levels to nanomolar concentrations of CRP, has been detected in a single immuno-assay in human serum. The median/average CVs were 9.9%/10.6% and 11.6%/13.1% for buffer and chicken serum, respectively, with a minimum CV of 3.8%

To quantitively compare our measurements to a gold standard method for biomarker measurement, the four analytes were quantified in each serum sample using Luminex panels (FIG. 6, right, Table 4). Since Luminex does not offer or recommend any one panel that can simultaneously measure all four analytes, each human serum sample was measured in three different panels: one for CRP, one for GDF-15, and a third for IL-1ra and IL-6 (see Methods). Notably, Luminex recommends different dilutions for each panel: 1,000-, 100-, and 3-fold for the CRP, GDF-15, IL-1ra/IL-6 panels, respectively. At the recommended dilutions for IL-6, two of the three human serum samples were below the limit of detection for Luminex, while EVROS was able to measure IL-6 as being within the expected physiological concentration range for all three serum samples. The concentrations of CRP reported by the Luminex assay were inordinately high; the measured range of ˜200 nM-2.8 μM CRP is near or above the highest reported clinical concentration of CRP observed in end-stage renal failure patients (433 nM) (Porazko, T. et al. IL-18 is involved in vascular injury in end-stage renal disease patients. Nephrol. Dial. Transplant. 24, 589-596 (2009); Nanjappa, V. et al. Plasma Proteome Database as a resource for proteomics research: 2014 update. Nucleic Acids Res. 42, 959-965 (2014)). Since samples were collected from healthy donors, this likely reflects inaccurate measurement by the Luminex test. The measurements for GDF-15 and IL-1ra were concordant between EVROS and Luminex, although both assays reported IL-1ra values that were lower than the reported healthy range. The results herein thus demonstrate that EVROS can produce robust, multiplexed measurements of analytes spanning the full proteomic physiological concentration range in undiluted biological specimens.

### CONCLUSION

In this work, EVROS—a methodology that makes it possible to simultaneously quantify both high- and low-abundance proteins from a single sample, with no differential dilution is introduced. This is achieved by employing a pair of tuning mechanisms—probe loading and epitope depletion—that make it possible to individually modulate the readout generated by each analyte in a molecular detection assay (in this demonstration, spPLA) and thereby bring those outputs into a dynamic range that can be quantitatively measured in a single assay. It was demonstrated this process for four targets present at physiological concentrations spanning more than seven orders of magnitude, which represents the greatest dynamic range achieved to date for multiplexed molecular analyte detection from a single measurement. In this work, it was demonstrated quantitative detection of analytes spanning high attomolar to nanomolar concentrations. No fundamental limitations with the tuning procedure has been encountered and believe it should be feasible to expand this range even further-certainly at the higher end of the concentration spectrum, and perhaps on the lower end as well. Critically, EVROS maintains robust performance in complex media, achieving results in undiluted chicken serum and human serum that are comparable to those obtained in buffer.

EVROS requires only 5 μL of sample, thereby greatly increasing the ability to study previously unattainable samples, including blood testing in neonates and even elderly patients. For example, in the context of neonatal care, only ˜700 μl/kg of whole blood can safely be extracted at any one time (˜6.3 mL/kg per month) (Garza, D. & Becan-McBride, K. Phlebotomy Handbook: Blood Collection Essentials, 7th Edition. (Pearson Prentice Hall, 2005))—a restriction that prevents neonates from being profiled with typical immunoassay workflows. Similarly, bio-banked samples are precious and limited in volume, and the ability to interrogate dozens, if not hundreds, of proteins from a single 5 μl sample would be a tremendous asset in this context.

Predictive heuristics were also introduced herein that simplify the development of future EVROS assays by enabling the rapid determination of optimum probe concentrations for a given target. The assay itself can also be scaled up substantially, from its current 96-well format to 384- or 1536-well formats. Beyond liquid handling, the multiplexing capacity of EVROS is limited only by the read-depth of the HTS system used; as sequencing technologies improve, so will the throughput of the EVROS assay.

### Supplemental Note 1|Derivation of Tuning Mechanism

A mechanistic model can be used to predict the tuning behavior of the EVROS system. The following assumptions were applied to equilibrium binding equations to derive the model: uniform K_{D} across the polyclonal pool, K_{D} does not change with increasing number of bound Abs, any Ab in the pool has equal likelihood of binding to any epitope on its intended target, and each target has n epitopes where n≥3. It is noted that these assumptions are too broad to predict behavior exactly but are enough to rationalize the trends observed.

To begin, n binding equations describing the equilibrium between i bound Abs and i+1 bound Abs,

$${K}_{D} = \frac{{[ {TA}_{i} ] [ A ]}_{tot} {f}_{Ab}}{[ {TA}_{i + 1} ]} for i = 0 , 1 , … , n - 1 , \qquad ( 1 )$$

$$where$$

$${f}_{Ab} = \frac{[ bound dAb ]}{[ total dAb ]} = 1 - \frac{{\sum }_{i = 1}^{n} i [ {TA}_{i} ]}{{[ A ]}_{tot}} , \qquad ( 2 )$$

[TAᵢ] is the concentration of target bound to i dAbs, [A]ₜₒₜ is the total concentration of dAbs, and f_{Ab} is the fraction of dAbs bound to target.

In this model, it was assumed that all dAbs are from the same species, and then later assigned these to a 3′ or 5′ designation based on probability. In the manuscript, equal concentrations were used of 3′- and 5′-dAbs; however, asymmetric concentrations of dAbs could also be used, and so fₚ was defined as the fraction of [dAb] that is the lower concentration. In other words, fₚ=0.5 for equivalent dAb concentrations, whereas if the 3′-dAb concentration is twice that of 5′-dAb, fₚ=0.33. The resulting signal can be defined using the binomial distribution and summing up the number of targets bound to multiple antibodies times the probability that there is a matching pair of 3′- and 5′-dAbs on each molecule:

$$S = {\sum }_{i = 2}^{n} {\sum }_{k = 1}^{i - 1} ( \begin{matrix}i \\ k\end{matrix} ) [ {TA}_{i} ] {( {f}_{p} ( 1 - {f}_{d} ) )}^{i - k} {( ( 1 - {f}_{p} ) ( 1 - {f}_{d} ) )}^{k} , \qquad ( 3 )$$

where fₚ is the asymmetry of the probes ([limiting probe]/[A]ₜₒₜ−[depletant]), f_{d} is the fraction of total antibody that is unlabeled ([depletant]/[A]ₜₒₜ), [TAᵢ] is the concentration of target that is bound to i dAbs, and n is the maximum number of binding epitopes on that target. Summing started at i=2 because fewer dAbs will not produce a signal. It was assumed that K_{D} is constant for subsequent binding events and is homogeneous (i.e., all Abs in the pool have the same K_{D}). The impact of this assumption is discussed later. Using Eq. 1-3, the distributions of the number of bound dAbs to the target molecules can be solved.

Increasing the concentration of dAb probes ([A]ₜₒₜ), or ‘probe loading’ will increase the number of DNA reporters that are produced, which is seemingly an undesired effect. However, this increase is asymmetric with respect to target concentration; the magnitude of this shift depends on [T], such that DNA outputs at low target concentrations are affected the least, whereas outputs at high target concentrations are strongly affected. The result is an upward and subtle rightward shift of the response curve as the amount of dAb increases. This upward shift can be offset using ‘epitope depletion’, achieved via the addition of unlabeled antibodies that compete with the DNA-labeled dAbs for the same target epitopes or by adding the two dAbs in different concentrations (i.e. asymmetric probe loading). This results in a fractional decrease in DNA production, shifting the response curve downward in a target concentration-independent fashion, as seen by the Eq. 3. The combined effects of probe loading and epitope depletion thus enable precise tuning of the dynamic range of the dose-response curve for each target individually.

For low-abundance proteins, our goal is to move the log-linear range of the signal response curve left or right by changing [A]ₜₒₜ. Assuming the dAbs exhibit a constant K_{D} across binding sites and that the concentration [T] of low-abundance proteins is much lower than the K_{D} of their associated antibodies, the binding curves of these proteins are given by:

$$f = \frac{[ T ]}{[ T ] + {K}_{D}} \approx \frac{[ T ]}{{K}_{D}} . \qquad ( 4 )$$

The signal of the assay is proportional to the concentration of target molecules that are bound to two dAbs, [TA₂], which can be described as a simple linear function of target concentration,

$$[ {TA}_{2} ] \approx \frac{{[ A ]}_{tot}^{2}}{{K}_{D}^{2}} [ T ] . \qquad ( 5 )$$

The log-linear range can be shifted up or down by changing [A]ₜₒₜ. Since the equation is linear over this range of target concentrations, this is equivalent to shifting the curve left or right:

$${log}_{10} [ {TA}_{2} ] = {log}_{10} [ T ] + {log}_{10} \frac{{[ A ]}_{tot}^{2}}{{K}_{D}^{2}} \qquad ( 6 )$$

### Supplemental Note 2|Predictive Tuning

It was generally observed that approximate predictions of reporter DNA output can be made independently of the target molecule. That is, despite the unpredictable effects of variables such as the number of available epitopes, the quaternary structure of the protein under assay conditions, and the average affinity of the polyclonal antibodies for their target, the same probe conditions will produce approximately the same DNA output response curves for different targets (FIG. 4). Therefore, a heuristic was developed that is only a function of (1) the concentration of the target and (2) the desired reporter DNA output concentration.

The heuristic works very well targets that are at concentrations below the average K_{D} of the antibodies (i.e., the binding curve isn't starting to saturate) and above the limit of detection of the assay. In this regime, the log-log response curves are almost completely linear and can be approximated by:

$$log [ DNA ] = α * log [ T ] + β * log [ probe ] + γ \qquad ( 7 )$$

where [T] is typically the log center of the target's physiological concentration range, [probe] is the concentration of each dAb, and [DNA] is the desired output reporter DNA copy number. Parameters α, β, and γ are scalar factors that are fit using scipy.optimize default parameters and empirical data from a few targets and probe concentrations. To estimate the probe concentration, solve for [probe] in Eq. 7, which gives this heuristic equation:

$$log [ probe ] = \frac{log [ DNA ] - α * {log [ T ]}^{*} - γ}{β} \qquad ( 8 )$$

Based on this approach, empirical data was used from the GDF-15 tuning shown in FIG. 4A, data from standard 250 pM dAb for all four targets shown in FIG. 4D, and unpublished IL-6 tuning data to fit for the parameters in our heuristic (α=0.85, β=1.46, and γ=−17.34). With these parameters, appropriate probe concentrations were estimated for IL-1ra and CRP (without depletant) to be 21 pM and 0.809 pM, respectively. The optimum IL-6 probe concentration was tuned to 372 pM. The resultant signals were within a two-fold range of the target output of 1 fM reporter DNA (FIG. 4E). This confirms that the heuristic was successful in producing good estimates of probe concentration without the need for laborious tuning.

### Supplemental Note 3|Hook Effect and Capture Bead Concentration

It is prudent to mention the well-known ‘hook effect’, where high target concentrations lead to decreased signal. It is accurately predicted by Eq. 3 (Supplemental Note 1) that target concentrations greatly exceeding [dAb] make it improbable that two detection probes will localize on the same target, resulting in a loss of signal. However, this was not observed in the data provided herein. For low-abundance proteins, the target concentrations are far below [dAb] and therefore never in the hook effect regime. And for high-abundance targets, the maximum concentration is imposed by the cAb density—once the beads are saturated, any excess target is washed away—so as long as [cAb]<[dAb], the hook effect is not observed for high abundance targets either. Nevertheless, this effect is still important to consider during assay design.

TABLE 2
Targets in our four-plex panel. For CRP, IL-1ra, and IL-6,
concentration ranges were estimated from the Plasma Proteome
Database, taking the highest and lowest reported values across
both plasma and serum, and excluding concentration values
taken from patients with severe disease. GDF-15 concentrations
were estimated from various R&D ELISA kit manuals.

| | | | Clinical | Approximate | |
|---|---|---|---|---|---|
| | | | Implication | Concentration | |
| | Molecular | | Clinical | Range | |
| Analyte | Weight | | Implication | (EVROS Target) | |
| C-reactive | 120 | kDa | Heart-related | ~0.1-36 | nM |
| protein | | | diseases, colon | | |
| (CRP) | | | cancer, general | | |
| | | | systemic | | |
| | | | inflammation | | |
| Growth/ | 24.6 | kDa | Various diseases | ~5-120 | pM |
| differentiation | | | including cancer, | | |
| factor 15 | | | diabetes, and | | |
| (GDF-15) | | | cardiovasular- | | |
| | | | related diseases | | |
| Interleukin | 25 | kDa | Inflammatory | ~0.8-60 | pM |
| 1 receptor | | | diseases involving | | |
| agonist | | | intestines, lungs, | | |
| (IL-1ra) | | | kidneys, and | | |
| | | | systemic infectious | | |
| | | | diseases. | | |

| | | | | |
|---|---|---|---|---|
| Interleukin | 21 | kDa | many inflammatory | undetected - |
| 6 (IL-6) | | | and autoimmune | 480 fM |
| | | | diseases including | |
| | | | Alzheimer's | |
| | | | Disease, cancer, | |
| | | | rheumatoid arthritis, | |
| | | | and major | |
| | | | depressive disorder. | |

TABLE 3
Assay conditions.

| | Final Probe | | | |
|---|---|---|---|---|
| | Concentration | Depletant | Beads per | [cAb] |
| Target | (pM) | (nM) | sample | (nM) |
| CRP | 3.93 | 5 | 7000000 | 1330 |
| GDF-15 | 10 | N/A | 700000 | 50 |
| IL-1ra | 21.6 | N/A | 700000 | 50 |
| IL-6 | 372 | N/A | 700000 | 50 |
| GFP | 10 | N/A | 700000 | 50 |

TABLE 4
Results from human serum.

| | H1 | | | H2 | | | H3 | | |
|---|---|---|---|---|---|---|---|---|---|
| | Avg | std | n | Avg | std | n | Avg | std | n |
| | | | | | EVROS | | | | |
| CRP | 16179 | 2482 | 3 | 73045 | 38720 | 3 | 16384 | 3105 | 3 |
| GDF-15 | 42.57 | 8.10 | 3 | 78.85 | 9.27 | 3 | 46.93 | 4.00 | 3 |
| IL-1ra | 5.92 | 1.35 | 3 | 15.23 | 1.58 | 3 | 8.38 | 0.16 | 3 |
| IL-6 | 0.08 | 0.03 | 3 | 0.29 | 0.13 | 3 | 5.20 | 0.59 | 3 |
| | | | | | Luminex | | | | |
| CRP | 419721 | 5977 | 2 | 2792351 | 102264 | 2 | 201502 | 13876 | 2 |
| GDF-15 | 14.37 | 0.85 | 2 | 19.16 | 1.70 | 2.00 | 10.48 | 0.42 | 2 |
| IL-1ra | 0.81 | 0.01 | 2 | 1.66 | 0.12 | 2.00 | 0.99 | 0.11 | 2 |
| IL-6 | n.d. | | 2 | 0.14 | 0.03 | 2.00 | n.d. | | 2 |

TABLE 5
Sequences used in work.

| | | Protein | Hyb | |
|---|---|---|---|---|
| Target | Name | Tag | Oligo | Oligos |
| IL-1ra | 3′ | TGAG | CCTAT | /5AmMC6/AAAAATCGTCGGCAGCGTCAGATGTGTATAAGAGACAG |
| | detection | GC | CCCTG | (N:25252525)(N)(N)(N)(N)(N)(N)(N)(N)(N)TGAGGC |
| | probe | (SEQ ID | (SEQ ID | CCTATCCCTG (SEQ ID NO: 2) |
| | | NO: 25) | NO: 1) | |
| IL-1ra | 5′ | CCCG | AGTCT | /5Phos/AGTCTATATGCCCGAT(N:25252525)(N)(N)(N)(N)(N) |
| | detection | AT | ATATG | (N)(N)(N)(N)CTGTCTCTTATACACATCTCCGAGCCCACGAG |
| | probe | (SEQ ID | (SEQ ID | ACAAAAA/3AmMO/ (SEQ ID NO: 4) |
| | | NO: 26) | NO: 3) | |
| IL-6 | 3′ | CGTG | TATCC | /5AmMC6/AAAAATCGTCGGCAGCGTCAGATGTGTATAAGAGACAG |
| | detection | GG | CTGAG | (N:25252525)(N)(N)(N)(N)(N)(N)(N)(N)(N)CGTGGGT |
| | probe | (SEQ ID | (SEQ ID | ATCCCTGAG (SEQ ID NO: 6) |
| | | NO: 27) | NO: 5) | |
| IL-6 | 5′ | GACCT | TCTAT | /5Phos/TCTATATGATGACCTC(N:25252525)(N)(N)(N)(N)(N) |
| | detection | C(SEQ | ATGAT | (N)(N)(N)(N)CTGTCTCTTATACACATCTCCGAGCCCACGAG |
| | probe | ID NO: | (SEQ ID | ACAAAAA/3AmMO/ (SEQ ID NO: 8) |
| | | 28) | NO: 7) | |
| GDF- | 3′ | CTGCC | TATGA | /5AmMC6/AAAAATCGTCGGCAGCGTCAGATGTGTATAAGAGACAG |
| 15 | detection | G(SEQ | TGACT | (N:25252525)(N)(N)(N)(N)(N)(N)(N)(N)(N)CTGCCGT |
| | probe | ID NO: | (SEQ ID | ATGATGACT (SEQ ID NO: 10) |
| | | 29) | NO: 9) | |
| GDF- | 5′ | GCGA | ATCGT | /5Phos/ATCGTACCCTGCGACG(N:25252525)(N)(N)(N)(N)(N) |
| 15 | detection | CG | ACCCT | (N)(N)(N)(N)CTGTCTCTTATACACATCTCCGAGCCCACGAG |
| | probe | (SEQ ID | (SEQ ID | ACAAAAA/3AmMO/ (SEQ ID NO: 12) |
| | | NO: 30) | NO: 11) | |
| CRP | 3′ | TCTGG | ATGAT | /5AmMC6/AAAAATCGTCGGCAGCGTCAGATGTGTATAAG |
| | detection | G(SEQ | GACTA | AGACAG(N:25252525)(N)(N)(N)(N)(N)(N)(N)(N)(N) |
| | probe | ID NO: | (SEQ ID | TCTGGGATGATGACTA (SEQ ID NO: 14) |
| | | 31) | NO: 13) | |
| CRP | 5′ | TAGC | TCGTA | /5Phos/TCGTACCCTGTAGCAC(N:25252525)(N)(N)(N)(N)(N) |
| | detection | AC | CCCTG | (N)(N)(N)(N)CTGTCTCTTATACACATCTCCGAGCCCACGAG |
| | probe | (SEQ ID | (SEQ ID | ACAAAAA/3AmMO/ (SEQ ID NO: 16) |
| | | NO: 32) | NO: 15) | |
| GFP | 3′ | GTCTA | ATCCC | /5AmMC6/AAAAATCGTCGGCAGCGTCAGATGTGTATAAGAGACAG |
| | detection | C(SEQ | TGAGT | (N:25252525)(N)(N)(N)(N)(N)(N)(N)(N)(N)GTCTACA |
| | probe | ID NO: | (SEQ ID | TCCCTGAGT (SEQ ID NO: 18) |
| | | 33) | NO: 17) | |
| GFP | 5′ | TAGAT | CTATA | /5Phos/CTATATGATGTAGATA(N:25252525)(N)(N)(N)(N)(N) |
| | detection | ID NO: | TGATG | (N)(N)(N)(N)CTGTCTCTTATACACATCTCCGAGCCCACGAG |
| | probe | A(SEQ | (SEQ ID | ACAAAAA/3AmMO/ (SEQ ID NO: 20) |
| | | 34) | NO: 19) | |

| Name | Sequence |
|---|---|
| Hyb | CAGGG/ideoxyU/ACGA/ideoxyU/AG/ideoxyU/CA/ideoxyU/CA/ideoxyU/A/ideoxyU |
| Oligo | /AGAC/ideoxyU/CAGGGA/ideoxyU/AGG (SEQ ID NO: 21) |
| Univ RP | GTC TCG TGG GCT CGG AGA T (SEQ ID NO: 22) |
| Univ FP | TCG TCG GCA GCG TCA GAT (SEQ ID NO: 23) |
| CTRL | AAAAATCGTCGGCAGCGTCAGATGTGTATAAGAGACAGNNNNNNNNNNTGTAGAGATG |
| Oligo | ACTATCGTACCCTGCACGGTACNNNNNNNNNNCTGTCTCTTATACACATCTCCGAGCCC |
| | ACGAGACAAAAA (SEQ ID NO: 24) |
| Modification | |
| Code | Modification Full Name |
| /5AmMC6/ | 5′ Amino Modifier C6 |
| /5Phos/ | 5′ Phosphorylation |
| /3AmMO/ | 3′ Amino Modifier |
| N:25252525 | Unique Hand mix ratio 25% per nucleotide |
| /ideoxy U/ | Int deoxy Uridine |

All patents, patent publications, patent applications, journal articles, books, technical references, and the like discussed in the instant disclosure are incorporated herein by reference in their entirety for all purposes.

It is to be understood that the figures and descriptions of the disclosure have been simplified to illustrate elements that are relevant for a clear understanding of the disclosure. It should be appreciated that the figures are presented for illustrative purposes and not as construction drawings. Omitted details and modifications or alternative embodiments are within the purview of persons of ordinary skill in the art.

It can be appreciated that, in certain aspects of the disclosure, a single component may be replaced by multiple components, and multiple components may be replaced by a single component, to provide an element or structure or to perform a given function or functions. Except where such substitution would not be operative to practice certain embodiments of the disclosure, such substitution is considered within the scope of the disclosure.

The examples presented herein are intended to illustrate potential and specific implementations of the disclosure. It can be appreciated that the examples are intended primarily for purposes of illustration of the disclosure for those skilled in the art. There may be variations to these diagrams or the operations described herein without departing from the spirit of the disclosure. For instance, in certain cases, method steps or operations may be performed or executed in differing order, or operations may be added, deleted or modified.

Where a range of values is provided, it is understood that each intervening value, to the smallest fraction of the unit of the lower limit, unless the context clearly dictates otherwise, between the upper and lower limits of that range is also specifically disclosed. Any narrower range between any stated values or unstated intervening values in a stated range and any other stated or intervening value in that stated range is encompassed. The upper and lower limits of those smaller ranges may independently be included or excluded in the range, and each range where either, neither, or both limits are included in the smaller ranges is also encompassed within the technology, subject to any specifically excluded limit in the stated range. Where the stated range includes one or both of the limits, ranges excluding either or both of those included limits are also included.

In the foregoing description, numerous specific details are set forth to provide a more thorough understanding of the present invention. However, it will be apparent to one of skill in the art that the invention described in this disclosure may be practiced without one or more of these specific details. In other instances, well-known features and procedures well known to those skilled in the art have not been described in order to avoid obscuring the invention. Embodiments of the disclosure have been described for illustrative and not restrictive purposes. Although the present invention is described primarily with reference to specific embodiments, it is also envisioned that other embodiments will become apparent to those skilled in the art upon reading the present disclosure, and it is intended that such embodiments be contained within the present inventive methods. Accordingly, the present disclosure is not limited to the embodiments described above or depicted in the drawings, and various embodiments and modifications can be made without departing from the scope of the claims below.

1. A method of tuning a proximity reporter assay comprising,
(a) providing a first series of compositions comprising first binding agents comprising a 5′ nucleic acid molecule and second binding agents attached to a 3′ nucleic acid molecule, wherein compositions in the first series comprise the first and second binding agents at different concentrations from the other compositions in the first series, and wherein the first and second binding agents bind a first target analyte;
(b) providing a second series of compositions comprising third binding agents comprising a 5′ nucleic acid molecule and fourth binding agents comprising a 3′ nucleic acid molecule, wherein compositions in the second series comprise the third and fourth binding agents at different concentrations from the other compositions in the second series, and wherein the third and fourth binding agents bind a second target analyte;
(c) forming a plurality of mixtures by contacting each composition from the first and second series of compositions with the first and second target analytes;
(d) performing, for each mixture in the plurality of mixtures, a proximity assay, wherein the assay produces, for each mixture, a signal comprising a nucleic acid product of the 5′ and 3′ nucleic acid molecules, wherein a first signal is produced for the first target and a second signal is produced for the second target; and
(e) selecting from each of the first and second series of compositions a single composition comprising binding agents at a concentration that results in first and second signals within four orders of magnitude for the first and second target analytes.

2. The method of claim 1, wherein the first and second signals are within three, two, or one orders of magnitude for the first and second target analytes.

3. The method of claim 1, wherein the first and second target analytes are present in the mixtures at a first and second concentration, respectively, and wherein the first and second concentrations vary by at least three orders of magnitude.

4. The method of claim 1, wherein the first and second concentrations vary by at least four, five, six, seven, eight, nine, ten, eleven or twelve orders of magnitude.

5. The method of claim 1, wherein each composition in the second series of compositions further comprises unlabeled binding agents that bind the second target analytes.

6. The method of claim 5, wherein the second target analyte is a high abundance target analyte.

7. The method of claim 1, wherein the first target analyte is a low or medium abundance target analyte.

8. The method of claim 1, wherein the first compositions further comprise first capture binding agents that bind the first analytes.

9. The method of claim 8, wherein the first capture binding agents are attached to a solid surface and wherein the solid surface is a bead.

10. (canceled)

11. The method of claim 1, wherein the second compositions further comprise second capture binding agents that bind the second analytes.

12. The method of claim 11, wherein the second capture binding agents are attached to a solid surface and wherein the solid surface is a bead.

13. (canceled)

14. The method of claim 1, wherein each mixture comprises both of the first and second target analytes.

15. The method of claim 1, wherein the proximity assay is a proximity ligation assay, and wherein the proximity ligation assay comprises ligating the 5′ and 3′ nucleic acids thereby producing the nucleic acid product comprising the 5′ and 3′ nucleic acids.

16. The method of claim 15, wherein the proximity assay further comprises amplifying the nucleic acid products or performing qPCR.

17. The method of claim 16, wherein the proximity assay further comprises sequencing the amplified nucleic acids products.

18. The method of claim 17, wherein the amplified nucleic acid products comprise a barcode sequence or a unique molecular identifier.

19. (canceled)

20. (canceled)

21. The method of claim 1, wherein the binding agents are antibodies, aptamers, nanobodies or combinations thereof.

22. (canceled)

23. The method of claim 1, wherein the mixtures of step (c) comprises both first and second analytes.

24. The method of claim 1, wherein the first series of compositions comprises 2, 3, 4, 5, 10, 15, 20, 25, 30, 35, 40, 45, 50 or more compositions and wherein the second series of compositions comprises 2, 3, 4, 5, 10, 15, 20, 25, 30, 35, 40, 45, 50 or more compositions.

25. (canceled)

26. The method of claim 1, further comprising repeating steps (a)-(e) one or more times.

27. The method of claim 1, further comprising performing a proximity assay using the selected compositions from step (e) by contacting the selected compositions with a biological sample comprising the first and second target analytes and determining the concentrations of the first and second target analytes in the biological sample based on the assay.

28. A method of providing a tuned proximity reporter assay comprising,
(a) providing an N series of compositions for N analytes, wherein each series of compositions corresponds to a different analyte, wherein each series comprises compositions comprising first binding agents comprising a 5′ nucleic acid molecule and second binding agents attached to a 3′ nucleic acid molecule for one analyte in the N sets of analytes, wherein the compositions in each series comprise the first and second binding agents at different concentrations from the other compositions in the series;
(b) for each N series of compositions, forming a plurality of mixtures by contacting each composition from the series of compositions with the analyte from the N analytes that corresponds to the series of compositions;
(c) performing, for each mixture in the plurality of mixtures, a proximity assay, wherein the assay produces, for each mixture, a signal comprising a nucleic acid product of the 5′ and 3′ nucleic acid molecules, wherein each signal corresponds to an analyte; and
(d) selecting for each N series of compositions a single composition comprising binding agents at a concentration that results in signals within four orders of magnitude for each analyte in the N analytes,
wherein the selected compositions from the N series of compositions provides a multitude of compositions each composition comprising the selected concentration of binding agents for each analyte within the N analytes, thereby providing a tuned proximity reporter assay.
